(19) United States
(12) Patent Application Publication
(10) Pub. No.: US 2020/0206187 A1
(43) Pub. Date: Jul. 2, 2020

(54) SCREENING PLATFORM TO IDENTIFY THERAPEUTIC DRUGS OR AGENTS FOR TREATMENT OF ALZHEIMER'S DISEASE

(71) Applicants: Rudolph E. TANZI, Cohasset, MA (US); Luisa QUINTI, Boston, MA (US); Doo Yeon KIM, Boston, MA (US)

(72) Inventors: Rudolph E. TANZI, Cohasset, MA (US); Luisa QUINTI, Boston, MA (US); Doo Yeon KIM, Boston, MA (US)

(21) Appl. No.: 16/628,918

(22) PCT Filed: Jan. 18, 2018
(86) PCT No.: PCT/US2018/014220

§ 371 Date: Jan. 6, 2020

Related U.S. Application Data
(60) Provisional application No. 62/530,125, filed on Jul. 8, 2017.
(60) Provisional application No. 62/535,589, filed on Jul. 21, 2017.

(51) Int. Cl. A61K 31/397; C07K 16/28

(52) U.S. Cl. CPC A61K 31/397; A61K 38/00; C07K 16/2803

(57) ABSTRACT

Disclosed are methods and compositions for screening candidate substances for the prevention or treatment of neurodegenerative disorders such as Alzheimer's disease. The disclosure also relates to identifying the mechanisms of action for known or suspected Alzheimer's disease drugs and generally to compositions and methods for modulating the function of cells expressing CD33.

### RELATED APPLICATIONS

This application claims benefit under 35 U.S.C. § 119(e) of the U.S. Provisional Application No. 62/530,125, filed Jul. 8, 2017 and the U.S. Provisional Application No. 62/535,589, filed Jul. 21, 2017, the contents of each of which are incorporated herein by reference in their entireties.

### TECHNICAL FIELD

This disclosure is related generally to methods and compositions for screening candidate substances for the prevention or treatment of neurodegenerative disorders. The disclosure also relates generally to compositions and methods for modulating the function of cells expressing CD33.

### BACKGROUND

Neurodegenerative diseases are a serious, common and growing worldwide problem. These include Alzheimer's disease (AD), cognitive impairment, Parkinson's disease, dementia, schizophrenia, amyotrophic lateral sclerosis (ALS), Huntington's disease or multiple sclerosis. Particularly in the elderly, neurodegenerative diseases cause suffering, reduced quality of life and are a major predictor of mortality. Alzheimer's disease (AD) is the most common form of neurodegenerative disease in the elderly and with an increasing population of the elderly in the US and worldwide, AD is reaching epidemic proportions. AD is characterized by progressive dementia and personality dysfunction. The abnormal accumulation of amyloid plaques in the vicinity of degenerating neurons and reactive astrocytes is a pathological characteristic of AD.

Microglia are the brain resident immune cells, responsible for clearing toxic pathogens, such as amyloid-β (Abeta, Aβ). During AD progression, microglia transition from a neuroprotective/pro-phagocytic to a neurotoxic state, characterized by increased secretion of pro-inflammatory cytokines (Heneka et al. Nature Immunology, 2015). It was found that microglial receptor CD33 is a late-onset AD risk factor in a large family-based GWAS analysis (Bertram et al., Am. J. Hum. Genet., 2008). It was also discovered that CD33 promoted amyloid pathology by inhibiting uptake and clearance of Aβ in microglial cells (Griciuc et al., Neuron, 2013).

Drugs such as donepezil and memantine treat the symptoms of AD but they do not treat the disease itself. A new drug, T-817MA (1-{3-[2-(1-Benzothiophen-5-yl)ethoxy]propyl}-3-azetidinol maleate) is in phase 1 and 2 clinical trials and it's neuroprotective effects against toxicity from Aβ and actions promoting neurite outgrowth are well documented (Takamura et al., Neurobiol Aging, 2014). How these drugs interact and modulate the activity of CD33 is unknown. Additionally, there remains a need to discover new treatments for neurodegenerative diseases such as AD. For example, rapid screening methods that can be applied to biologically relevant compounds and combinations of compounds, and their dose response would provide a significant advance in this field and lead to break-through therapies and drugs that can help eradicate and control these diseases.

### SUMMARY

The present disclosure relates to the discovery of methods for screening candidate substances for the prevention or treatment of neurodegenerative disorders. The disclosure also relates to the discovery of the microglial receptor CD33 as a risk factor in neurodegenerative diseases and to compositions and methods for modulating the function of cells expressing CD33 as a screening assay and mechanistic probe.

In one aspect, there is provided a method for testing a therapeutic efficacy of a candidate substance for a prevention or treatment agent of a neurodegenerative disorder. Generally, the method comprises treating immune or immune-like cells expressing full-length human CD33, also referred to as CD33 expressing immune or immune-like cells herein, with a candidate substance at a relevant concentration, further treating said treated CD33 expressing immune or immune-like cells with amyloid-β (Abeta, Aβ) at a relevant concentration or lipopolysaccharide, and measuring intracellular levels of Aβ (for Aβ treated cells) or culture media levels of pro-inflammatory cytokines (for lipopolysaccharide treated cells). The CD33 expressing immune or immune-like cells optionally can be cultured prior to treatment with the candidate substance.

In embodiments of the method comprising treatment with Aβ, higher levels of Aβ in CD33 cells treated with the candidate substance, relative to a negative control, indicate that the tested candidate substance has therapeutic efficacy. In embodiments of the method comprising treatment with lipopolysaccharide, lower levels of one or more pro-inflammatory cytokines in the culture media of CD33 cells treated with the candidate substance, relative to a negative control, indicate that the tested candidate substance has therapeutic efficacy.

In some embodiments, the method comprises treating CD33 expressing immune or immune-like cells with a candidate substance at a relevant concentration; further treating said treated CD33 expressing immune or immune-like cells with amyloid-β (Abeta, Aβ) at a relevant concentration; and measuring intracellular levels of Aβ in said CD33 expressing cells, wherein higher levels of Aβ in CD33 cells treated with the candidate substance, relative to a negative control, indicate that the tested candidate substance has therapeutic efficacy.

In some embodiments, the method comprises treating CD33 expressing immune or immune-like cells with a candidate substance at a relevant concentration; further treating said treated CD33 expressing immune or immune-like cells with lipopolysaccharide; and measuring levels of pro-inflammatory cytokines in culture media of said CD33 expressing cells, wherein lower levels of pro-inflammatory cytokines in the culture media of CD33 cells treated with the candidate substance, relative to a negative control, indicate that the tested candidate substance has therapeutic efficacy.

In another aspect, there is provided a method for testing a binding interaction of a substance with human CD33. Generally, the method comprises treating said CD33 expressing immune or immune-like cells with a candidate substance at a relevant concentration; and measuring a read-out of the binding interaction using a standard assay, wherein a higher read-out in CD33 cells treated with the substance, relative to a negative control, indicate a binding interaction. The CD33 expressing immune or immune-like cells optionally can be cultured prior to treatment with the candidate substance.

In some embodiments of the various aspects described herein, the CD33 expressing immune or immune-like cells are microglial cells.

In some embodiments of the various aspects described herein, the CD33 expressing immune or immune-like cells optionally can be cultured prior to treatment with the candidate substance.

The substances identified as having therapeutic efficacy or a binding interaction with CD33 can be used for modulating a function of a microglial cells. The microglial cell can be in vitro, in vivo, or ex vivo. Accordingly, in another aspect, provided herein is a method for modulating a function of a microglial cell in a subject. Generally, the method comprises administering to a patient or subject in need thereof a substance identified by a method described herein.

In some embodiments, the substance that is administered to the subject is identified as having therapeutic efficacy by a method described herein.

In some embodiments, the substance that is administered to the subject is 1-(3-(2-(1-benzothiophen-5-yl)ethoxy)propyl)azetidin-3-ol or a salt thereof.

In some embodiments, the method can be used for treating or preventing a neurodegenerative disease in a subject. For example, by administering to a patient or subject in need thereof a therapeutic dose or dosages of a substance identified by a method described herein. Exemplary neurodegenerative disorders include, but are not limited to, Alzheimer's disease, mild cognitive impairment, Parkinson's disease, dementia, schizophrenia, amyotrophic lateral sclerosis, Huntington's disease and multiple sclerosis. For example, the methods can be applied to Alzheimer's disease.

In another aspect, there is provided a pharmaceutical composition for modulating a function of microglial cells. Generally, the pharmaceutical composition comprises 1-(3-(2-(1-benzothiophen-5-yl)ethoxy)propyl)azetidin-3-ol, or a salt thereof.

In some embodiments, the pharmaceutical composition further comprises a pharmaceutically acceptable excipient or carrier.

In some embodiments of the various aspects described herein, the method enhances phagocytosis or inhibits cytokine production of microglial cells.

In some embodiments of the various aspects described herein, the compound is 1-(3-(2-(1-benzothiophen-5-yl)ethoxy)propyl)azetidin-3-ol maleate, also referred to as T-817MA herein.

Other features and advantages of the invention will be apparent from the Detailed Description, and from the Claims. Thus, other aspects of the invention are described in the following disclosure and are within the ambit of the invention.

### BRIEF DESCRIPTION OF THE DRAWINGS

FIG. 1 shows the results as a bar graph from a lactate dehydrogenase (LDH) toxicity assay on naïve BV2 microglial cells after 5 hours of treatment with a DMSO, 817MA, 817A11, and 614P. No statistically significant toxicity was seen at these concentrations.

FIG. 2 shows the results as a bar graph from an LDH toxicity assay on wtCD33-expressing microglial cells after 5 hours of treatment with a DMSO, 817MA, 817A11, and 614P. No statistically significant toxicity is seen at these concentrations.

FIG. 3 shows the results for an Abeta42 uptake assay into naïve BV2 microglial cells as a line graph. The results for DMSO, 817MA, 817A11 and 614P into naïve BV2 microglial cells is shown. Compounds 817MA and 817A11 have similar Abeta42 uptake EC₅₀s. Compound 614P has a higher EC₅₀.

FIG. 4 shows the results for an Abeta42 uptake assay into wt-CD33 expressing BV2 cells as a line graph. The results for DMSO, 817MA, 817A11 and 614P into wt-CD33 expressing BV2 cells is shown. Compounds 817MA and 817A11 have similar Abeta42 uptake EC₅₀s. Compound 614P has a higher EC₅₀.

FIG. 5 shows the results for an Abeta40 uptake assay into naïve BV2 microglial cells as a line graph. The results for DMSO, 817MA, 817A11 and 614P into naïve BV2 microglial cells is shown. Compound 817A11 has a lower uptake EC₅₀ than 817MA. Compound 614P has a higher EC₅₀ than both 817MA and 817A11.

FIG. 6 shows the results for an Abeta40 uptake assay into wt-CD33 expressing BV2 cells as a line graph. The results for DMSO, 817MA, 817A11 and 614P into wt-CD33 expressing BV2 cells is shown. Compounds 817A11 has a lower uptake EC₅₀ than 817MA. Compound 614P has a higher EC50 than both 817MA and 817A11.

FIG. 7 is a table showing LPS activation. Ten cytokines were simultaneously analyzed in microglial conditioned media. The cytokines KC/GRO, IL-6, IL-10 and TNF-α showed detectable levels upon LPS activation. KC/GRO could only be detected in undiluted media. Cytokines IFN-γ, IL-2, IL-5, IL-12p70, IL-10 and IL-4 were not detected.

FIG. 8 shows the results for a first experiment for BV2 LPS activation of TNFα as a line graph. The graph shows the TNFα concentration production response as a function of DMSO, 817MA, 817A11 and 614P added concentrations. Compound 817MA is the only compound effectively reducing TNFα production.

FIG. 9 shows the results for a second experiment for BV2 LPS activation of TNFα as a line graph. The graph shows the TNFα concentration production response as a function of DMSO, 817MA, 817A11 and 614P added concentrations. Compound 817MA is the only compound effectively reducing TNFα production.

FIG. 10 shows the results for a first experiment for BV2 LPS activation of IL-6 as a line graph. The graph shows the IL-6 concentration production response as a function of DMSO, 817MA, 817A11 and 614P added concentrations. Compound 817MA is the most effective compound for reducing IL-6 production.

FIG. 11 shows the results for a second experiment for BV2 LPS activation of IL-6 as a line graph. The graph shows the IL-6 concentration production response as a function of DMSO, 817MA, 817A11 and 614P added concentrations. Compound 817MA is the most effective compound for reducing IL-6 production.

FIG. 12 shows the results for a first experiment for BV2 LPS activation of IL-10 as a line graph. The graph shows the IL-10 concentration production response as a function of DMSO, 817MA, 817A11 and 614P added concentrations. Compound 817MA is the most effective compound for reducing IL-10 production.

FIG. 13 shows the results for a second experiment for BV2 LPS activation of IL-10 as a line graph. The graph shows the IL-10 concentration production response as a function of DMSO, 817MA, 817A11 and 614P added concentrations. Compound 817MA is the most effective compound for reducing IL-10 production.

FIG. 14 shows the results for a first experiment for BV2 LPS activation of KC/GRO as a line graph. The graph shows the KC/GRO concentration production response as a function of DMSO, 817MA, 817A11 and 614P added concentrations.

FIG. 15 shows the results for a second experiment for BV2 LPS activation of KC/GRO as a line graph. The graph shows the KC/GRO concentration production response as a function of DMSO and 817MA added concentrations.

FIG. 16 shows the results in a bar graph form for data from a first test in AD 3D ReN cell culture system. The test is and LDH assay in HReN30-mGAP30 media after one-week treatment with test compounds DMSO, T-817MA (817MA), T-817A11(817A11) and T-614 (614P). n is 3 or 4 for each cell line.

FIG. 17 shows the results in a bar graph form for data from a second test in AD 3D ReN cell culture system. The test is an LDH assay in HReN30-mGAP10# D4 media after one-week treatment with test compounds DMSO, T-817MA (817MA), T-817A11(817A11) and T-614 (614P). n is 3 or 4 for each cell line.

FIG. 18A-18H shows a series of bar graphs of data for soluble (media) (horizontal axis) and insoluble Abeta levels (vertical axis) after drug treatments. The test compounds are T-817MA (817MA), T-817A11(817A11) and T-614 (614P). FIG. 18A shows Abeta40 in HReN30 (HReN-mGAP30) Media. FIG. 18B shows Abeta42 in HReN30 (HReN-mGAP30) Media. FIG. 18C shows Abeta40 in HReN30 (HReN-mGAP30) insoluble fraction. FIG. 18D shows Abeta42 in HReN30 (HReN-mGAP30) insoluble fraction. FIG. 18E to 18H are ReN-mGAP # D4 experiments. FIG. 18E shows Abeta40 in ReN-mGAP10# D4 (ReN-mGAP # D4) Media. FIG. 18F shows Abeta42 in ReN-mGAP10# D4 (ReN-mGAP # D4) Media. FIG. 18G shows Abeta40 in ReN-mGAP10# D4 (ReN-mGAP # D4) insoluble fraction. FIG. 18H shows Abeta42 in ReN-mGAP10# D4 (ReN-mGAP # D4) insoluble fraction.

FIG. 19A to 19D shows a series of bar graphs of data for insoluble p-tau and total p-tau levels after drug treatments. The test compounds are DMSO, T-817MA (817MA), T-817A11(817A11) and T-614 (614P). FIGS. 19A and 19B are HReN-mGAP30 tests. FIG. 19A shows pTau181 concentration (unit/mL) in HReN30 insoluble fraction. FIG. 19B shows pTau181 concentration (pg/mL) in HReN30 insoluble fraction. FIG. 19C and FIG. 19D are ReN-mGAP # D4 tests. FIG. 19C shows pTau181 concentration (unit/mL) in ReN-mGAP10# D4 insoluble fraction. FIG. 19D shows pTau181 concentration (pg/mL) in ReN-mGAP10# D4 insoluble fraction.

FIG. 20 shows a series of images in ReN-mGAP # D4 (4-week differentiation).

FIG. 21 shows a series of images in HReN-mGAP30 (7-week differentiation).

FIG. 22 shows a bar graph of data for sodium nitroprusside (SNP) toxicity studies in an AD 3D ReN cell culture system. The data is of WST-8 assay in ReN cells without B27 after one-day treatment with SNP.

FIG. 23 shows a bar graph of data for a WST-8 assay in ReN cells without B27 after four days of 817MA and one day of SNP treatment.

FIG. 24 shows a bar graph of data for a WST-8 assay (% viability) in ReN cells without B27 after four days of 817MA and one day of SNP treatment.

FIG. 25 show a bar graph of data for a toxicity (LDH) assay in microglial cells. The test compound is 817MA at various concentrations and includes a DMSO control. The 50 μM concentration was excluded from the uptake analysis.

FIG. 26 shows a bar graph of data for an Abeta42 uptake assay. The data confirms uptake of Abeta42 by 817MA in microglial cells. EC50 is about 20 μM following 24 hours of compound pre-treatment.

FIGS. 27A and 27B show exemplary time line diagrams for 817MA treatments.

FIG. 28 shows a bar graph of data for cell viability to SNP concentration at day 26.

FIG. 29 shows a bar graph of data for cell viability at selected concentrations. n=3 or 4 for each cell line.

FIGS. 30A and 30B show bar graphs showing data for the 817MA effect on SNP-induced toxicity after 3 days (FIG. 30A) and after 3 weeks (FIG. 30B).

FIGS. 31A and 31B show bar graphs showing data for the 817MA effect on cell viability (no SNP) for 3 days (FIG. 31A) and for 3 weeks (FIG. 31B).

FIG. 32 shows a series of microscope images illustrating the SNP effect on cells.

FIG. 33 shows a second series of microscope images illustrating the SNP effect on cells.

FIG. 34 shows a bar graph of data for the effect of a four-day treatment with 817MA on SNP-induced toxicity in non-AD cells.

### DETAILED DESCRIPTION

The present disclosure is based, in part, on the discovery of valuable tools to study potential drugs for the treatment of neurodegenerative diseases or disorders. The disclosure is also based in part on producing cells expressing microglial receptors that are a risk factor in late-onset AD and that lead to amyloid pathology. It has been found as described herein that such cells can be used for screening potential drug candidates as therapeutics for neurodegenerative disorders. The cells also provide insight into the mechanism of drug activity and to the discovery of important features (e.g., structure to activity relationships) of effective drug treatments. In addition, the disclosure provides compositions that are effective in the treatment of neurodegenerative disorders and their effect on the CD33 expressing cells.

Accordingly, in one embodiment, there is provided a method for testing a therapeutic efficacy of a candidate substance for a prevention or treatment agent of a neurodegenerative disorder. Generally, the method comprises treating immune or immune-like cells expressing full-length human CD33 with a candidate substance at a relevant concentration. The method also includes treating the treated CD33 expressing immune or immune-like cells with amyloid-β at a relevant concentration, and measuring intracellular levels of Aβ. In an alternative embodiment to the Aβ treatment, the method includes treating the CD33 expressing immune or immune-like cells with lipopolysaccharide and measuring the levels of pro-inflammatory cytokines in a cultured media of these cells. The CD33 expressing immune or immune-like cells optionally can be cultured prior to treatment (Aβ or lipopolysaccharides) with the candidate substance.

A therapeutic refers to any drug, substance, compound, combination of compounds, treatment agent, or treatment therapy that is a used for the purpose of alleviating the symptoms of or curing a disease, condition or disorder. In some embodiments the therapeutic can be a test compound, for which the effectiveness of the compound is not known until the screening assay or test is completed.

Therapeutic efficacy relates to how effective a test compound (e.g., a substance, compound, combination of compounds, treatment agent, or treatment therapy) is. A test compound having therapeutic efficacy means it is more effective for alleviating the symptoms of or curing a disease or condition as compared a control. In addition, a high therapeutic efficacy of a test compound means it is more effective for alleviating the symptoms of or curing a disease or condition as compared to another test compound with a lower therapeutic efficacy. For example, the control can be a compound that has a lower therapeutic efficacy than a test compound with therapeutic efficacy, and the control is not as effective in alleviating the symptoms of or curing a disease or condition. The term “more effective” can include that a lower dosage of the therapeutic provides the same amount of benefit, has fewer undesirable, harmful or toxic side effects, or the more effective therapeutic has additional benefits (e.g., health benefits, cost benefits), as compared to the less effective therapeutic.

Toxicity and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices are preferred. As used herein, the term ED denotes effective dose and is used in connection with animal models. The term EC denotes effective concentration and is used in connection with in vitro models.

As used herein a “control” is a drug, substance, compound, compounds and/or test condition with a known therapeutic effect, such as no therapeutic effect or efficacy or some specific amount of therapeutic effect of efficacy. As used herein, a “negative control” can be a control that has similar physical characteristics to the test therapeutic composition but is known to have no therapeutic effect. For example, the negative control can be a solvent, diluent or delivery agent that the test compound is dissolved/combined with during the test, but as the negative control the test compound is excluded in/from the solvent, diluent or delivery agent. For example, the test compound can be any one or more of a solvent, DMSO, water, alcohol, a micelle, vesicle, protein, polymer or complexing agent. A “positive control” can be a control for which there is a known therapeutic effect and would therefore provide a positive result in a test for that therapeutic effect.

As used herein, the term “candidate compound”, “candidate substance”, “test compound” or “test agent” refers to any compound, molecule or agent that is to be tested. As used herein, the terms, which are used interchangeably, refer to biological or chemical compounds such as simple or complex organic or inorganic molecules, small molecules, peptides, proteins, oligonucleotides, polynucleotides, carbohydrates, or lipoproteins. A vast array of compounds can be synthesized, for example oligomers, such as oligopeptides and oligonucleotides, and synthetic organic compounds based on various core structures, and these are also included in the terms noted above. In addition, various natural sources can provide compounds for screening, such as plant or animal extracts, and the like. Compounds can be tested singly or in combination with one another. Agents or candidate compounds can be randomly selected or rationally selected or designed. As used herein, an agent or candidate compound is said to be “randomly selected” when the agent is chosen randomly without considering the specific interaction between the agent and the target compound or site. As used herein, an agent is said to be “rationally selected or designed”, when the agent is chosen on a nonrandom basis which takes into account the specific interaction between the agent and the target site and/or the conformation in connection with the agent's action. In some embodiments, the assays described herein can be used to guide a rational design, for example, by providing mechanistic insight into the efficacy of a test compound which is feed in an iterative fashion into a series of assays or screens while refining the selection of test compounds. A test compound can be a control compound.

As used herein, the term “small molecule” can refer to compounds that are “natural product-like,” however, the term “small molecule” is not limited to “natural product-like” compounds. Rather, a small molecule is typically characterized in that it contains several carbon-carbon bonds, and has a molecular weight more than about 50, but less than about 5000 Daltons (5 kD). Preferably the small molecule has a molecular weight of less than 3 kD, still more preferably less than 2 kD, and most preferably less than 1 kD. In some cases, it is preferred that a small molecule have a molecular mass equal to or less than 700 Daltons.

Depending upon the particular embodiment being practiced, the test compounds can be provided free in solution, or may be attached to a carrier, or a solid support, e.g., beads. A number of suitable solid supports may be employed for immobilization of the test compounds. Examples of suitable solid supports include agarose, cellulose, dextran (commercially available as, i.e., Sephadex, Sepharose) carboxymethyl cellulose, polystyrene, polyethylene glycol (PEG), filter paper, nitrocellulose, ion exchange resins, plastic films, polyaminemethylvinylether maleic acid copolymer, glass beads, amino acid copolymer, ethylene-maleic acid copolymer, nylon, silk, etc. Additionally, for the methods described herein, test compounds can be screened individually, or in groups. Group screening is particularly useful where hit rates for effective test compounds are expected to be low such that one would not expect more than one positive result for a given group. Group screening is also useful for determining hits that can act synergistically.

As used herein, the term “neurodegenerative disease” refers to a varied assortment of central nervous system disorders characterized by gradual and progressive loss of neural tissue and/or neural tissue function. A neurodegenerative disease is a class of neurological disorder or disease, and where the neurological disease is characterized by a gradual and progressive loss of neural tissue, and/or altered neurological function, typically reduced neurological function as a result of a gradual and progressive loss of neural tissue. Examples of neurodegenerative diseases include for example, but are not limited to, Alzheimer's disease (AD), Parkinson's disease (PD), Huntington's Disease, Amyotrophic Lateral Sclerosis (ALS, also termed Lou Gehrig's disease) and Multiple Sclerosis (MS), polyglutamine expansion disorders (e.g., HD, dentatorubropallidoluysian atrophy, Kennedy's disease (also referred to as spinobulbar muscular atrophy), spinocerebellar ataxia (e.g., type 1, type 2, type 3 (also referred to as Machado-Joseph disease), type 6, type 7, and type 17)), other trinucleotide repeat expansion disorders (e.g., fragile X syndrome, fragile XE mental retardation, Friedreich's ataxia, myotonic dystrophy, spinocerebellar ataxia type 8, and spinocerebellar ataxia type 12), Alexander disease, Alper's disease, ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, ischemia stroke, Krabbe disease, Lewy body dementia, multiple system atrophy, Pelizaeus-Merzbacher disease, Pick's disease, primary lateral sclerosis, Refsum's disease, Sandhoff disease, Schilder's disease, spinal cord injury, spinal muscular atrophy (SMA), SteeleRichardson-Olszewski disease, Tabes dorsalis, and the like. In some embodiments the disease is a subset of these diseases such as Alzheimer's disease, mild cognitive impairment, Parkinson's disease, dementia, schizophrenia, amyotrophic lateral sclerosis Huntington's disease or multiple sclerosis. In some other embodiments, the neurodegenerative disease is Alzheimer's disease.

As described herein “culturing cells” or “culturing a cell” refers to growing cells to increase their population. This can be done in a “cell culture medium” (also referred to herein as a “culture medium” or “medium”) which as referred to herein is a medium for culturing cells containing nutrients that maintain cell viability and support proliferation. The cell culture medium can contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art. In addition, for the assays and testing purposes as described herein, a cell culture can be 2D cell culture, such as a thin film or monolayer, or a 3D cell culture. A wide variety of techniques currently exist to culture cells into 3D structures. Without limitations, these 3D cell culture models can include polymeric hard scaffolds, biologic scaffolds, micropatterened surface microplates, hanging drop microplates, spheroid microplates containing Ultra-Low Attachment coatings or microfluidic 3D cell cultures. In some embodiments a AD 3D ReN cell culture system can be utilized, for example, as described in A 3D human neural cell culture system for modeling Alzheimer's disease, Y. H Kim et al., Nat Protoc. 2015 July; 10(7): 985-1006.

As used herein “immune” cells and “immune-like” cells are any of various cells that engulf, destroy or incapacitate pathogens. For example, cells that can function in an immune system by protecting against pathogens and aiding in tissue repair. These include white blood cells (e.g., leukocytes, white cell, white corpuscle), which are produced in bone marrow. Immune cells include neutrophils, macrophage, dendritic cells, eosinophils, basophils, lymphocytes, and monocytes—and can be found in blood, lymph, and other tissues. These can also include “microglial cells” which are resident cells of the central nervous system. In some embodiments the immortalized murine microglial cell line BV-2 is used.

CD33 is a transmembrane myeloid specific member of the sialic acid-binding receptor family and is expressed highly on myeloid progenitor cells but at much lower levels in differentiated cells. Binding of sialic acid activates CD33, leading to monocyte inhibition via immunoreceptor tyrosine-based inhibitory motif domains. Human CD33 has two tyrosine residues in its cytoplasmic domain (Y340 and Y358). In some embodiments CD33 can include the “full length” peptide. The amino acid sequence for CD33 is known in the art and is provided below for reference:

mplllllpll wagalamdpn fwlqvqesvt vqeglcvlvp ctffhpipyy dknspvhgyw fregaiisrd spvatnkldq evqeetqgrf rllgdpsrnn cslsivdarr rdngsyffrm ergstkysyk spqlsvhvtd lthrpkilip gtlepghskn ltcsyswace qgtppifswl saaptslgpr tthssvliit prpqdhgtnl tcqvkfagag vttertiqln vtyvpqnptt gifpgdgsgk qetragvvhg aiggagvtal lalcicliff ivkthrrkaa rtavgrndth pttgsaspkh qkksklhgpt etsscsgaap tvemdeelhy aslnfhgmnp skdtsteyse vrtq (SEQ ID NO: 1). Optionally, the truncated peptides lacking the sialic acid binding domain can be used in some embodiments.

In some embodiments the immortalized murine microglial cell line BV-2 are used. In some embodiments the BV-2 cells expressing full length human CD33 BV-2 cells are used while in other embodiments BV-2 cells expressing CD33 lacking sialic acid binding domain are used. In some embodiment of the assays described herein cells can be an isolated population of a substantially pure cells.

As used herein “treating” a cell with a substance means to contact the cell with the substance for any amount of time. For example, combining the cell and compound directly or combining them in a medium such as solvents, buffers or other media. For example, the media can include a cell growth media, biological fluids such as cerebrospinal fluid, blood or plasma, or simulated biological fluids. The treatment can be for any amount of time such as for between 1 second and several days such as 60 or more days. For example, treatment can be for between one minute and 60 days, between 1 hour and 45 days, between 1 day and 30 days, for between 1 and 7 days, between 1 and 3 days. Treatment can also include incubation (e.g., at temperatures between 5 and 50° C., between 25 and 40° C., about 37° C.), mixing, labeling, isolation, sonication, centrifugation, filtration, lyophilization and irradiation simultaneous with, prior to or following the treatment.

The test compound or therapeutic can be tested at any desired concentration. As used herein “relevant concentration” refers to a concentration that is close to a concentration that is expected to have an effect and can be formulated into a drug for administration to a subject. For example, the test compound can be tested at a final concentration of from 0.01 nM to about 10 mM. Further, the test can be tested at 2 or more (e.g., 2, 3, 4, 5, 6, 7, 8, 9, 10 or more) different concentrations. This can be helpful if the test compound is active only in a range of concentration. When the test compound is tested at 2 or more different concentrations, the concentration difference can range from 10-10,000 fold (e.g., 10-5000 fold, 10-1000 fold, 10-500 fold, or 10-250 fold). In addition, two or more different compounds can be tested simultaneously or added sequentially in any combination of order and concentrations.

As used herein “amyloid-β” or “β-Amyloid peptide,” (Aβ or Abeta) are a group of peptides 36-43 amino acids in length that are the main component of the sticky buildup called amyloid plaques found in the brains of AD patients. The peptides can be derived from the amyloid precursor protein (APP), which is cleaved by beta secretase and gamma secretase to yield Aβ. The two major isoforms of Aβ are: the 42-residue Aβ42 (Abeta42) and the 40-residue Aβ40 (Abeta40). Aβ42 has two extra residues at the C-terminus as compared to Aβ40. The amyloid plaques in Alzheimer's brains can consist of mostly Aβ42 and some plaques contain only Aβ42, even though vascular Aβ40 concentration is several-fold more than Aβ42. The Aβ as relates in the embodiments can be both of a natural or synthetic form.

As used herein a “lipopolysaccharide” is a compound in which a lipid molecule is bound to a polysaccharide by a covalent bond. For example, Endotoxin which can refer to any cell-associated bacterial toxin or can refer to the complex associated with the outer membrane of Gram-negative pathogens such Escherichia coli, Salmonella, Shigella, Pseudomonas, Neisseria, Haemophilus influenzae, Bordetella pertussis and Vibrio cholera. The term can refer to a molecule including a hydrophobic lipid section, a hydrophilic core polysaccharide, and a repeating hydrophilic O-antigenic oligosaccharide side chain. The lipid section can be made up of a β-glucosamine-(1→6)-glucosamine-1-phosphate base with fatty acid esters attached to both carbohydrates. The hydrophilic core polysaccharide can include an inner and outer core. the inner polysaccharide core typically contains between 1 and 4 molecules of the KDO (3-deoxy-α-D-manno-octulosonic acid) attached to the disaccharide core. The KDO-containing inner core can also be modified with heptulose (ketoheptose) monosaccharides, the most common of which is L-glycero-α-D-manno-heptopyranose. The inner core glycan residues can be phosphorylated or modified with phosphate-containing groups, e.g., pyrophosphate or 2-aminoethylphosphate. The outer core of the lipopolysaccharide can include more common hexoses, including glucose, galactose, and N-acetylglucosamine and can be structurally more diverse than the inner core. The O-antigen is a repeating oligosaccharide unit typically comprised of two to six sugars.

As used herein “cytokines” refers to small proteins such as can be released by cells and effect the interactions and communications between cells. Cytokine include lymphokine (cytokines made by lymphocytes), monokine (cytokines made by monocytes), chemokine (cytokines with chemotactic activities), and interleukin (cytokines made by one leukocyte and acting on other leukocytes). Cytokines may act on the cells that secrete them (autocrine action), on nearby cells (paracrine action), or on distant cells (endocrine action). There are both pro-inflammatory cytokines (such as TNFα, IL1, IL6, IL8) and anti-inflammatory cytokines such as (TGF-β and IL-10). In some embodiments cytokines can include, but are not limited to, IFN-γ, IL-2, IL-5, IL-12p70, IL-10, IL-4, KC/GRO, IL-6, IL-10 and TNF-α.

By “measuring” as used herein such as in measuring intracellular levels or measuring levels can mean a qualitative, semi-quantitative, or quantitative measurement method. For example, a qualitative measurement can include the detection of the presence or absence of an indicator such as can be detected by the presence or absence of a color in a sample. In some embodiments, the qualitative measurement detects the presence or absence of Aβ or pro-inflammatory cytokines, e.g., through an indicator molecule or tag. A semi-quantitative method can include the ranking of two or more samples for example, from highest to lowest- or more intense to less intense, with respect to a color indicator. A quantitative measurement can include a numerical value of the concentration of an analyte in the sample. In some embodiments the measurement provides the concentration of Aβ and pro-inflammatory cytokines in the test sample. In some embodiments the measurement is of absorbance, fluorescence or % cell viability. For example, the enzyme-linked immunosorbent assay (ELISA) can be used for quantitating Aβ such by using the Amyloid beta 40 Human ELISA Kit or the Amyloid beta 42 Human ELISA Kit commercially available (Thremo Scientific). In some embodiments the methods include measuring the toxicity of a test compound. For example, the toxicity can be tested utilizing a cytotoxicity (LDH) test, such as a Pierce™ LDH cytotoxicity Assay Kit (Thermo Scientific) or CytoTox-ONE™ LDH assay (Promega, WI). In some embodiments, the methods include measurement on cytokines. ELISA can be used for measuring cytokines, as can variants of ELISA which use a surface such as an addressable bead (e.g., Luminex® Mulitiplex Assays, Invitrogen-thermofisher).

As used herein the “T-817” or “817” refers to the compound 1-(3-(2-(1-benzothiophen-5-yl)ethoxy)propyl)azetidin-3-ol. The compound “T-817MA” or “817MA” refers to edonerpic, or edonerpic maleate which is 1-(3-(2-(1-benzothiophen-5-yl)ethoxy)propyl)azetidin-3-ol maleate.

As used herein “T-817A11” or “817A11” refers to 1-{3-[2-(1-benzothiophen-5-yl)ethoxy]propionyl}azetidin-3-ol.

As user herein “T-614P” and “614P” and refer the compound Iguratimod having the chemical name N-(3-formamido-4-oxo-6-phenoxy-4H-chromen-7-yl).

As used herein a “binding interaction” or “binding affinity” is a quantitative or qualitative measure of the strength of the binding interaction between two substances such a two proteins, a protein-small molecule, or a protein and a nucleic acid. Binding affinity can be measured and reported as the equilibrium dissociation constant (K_{D}), which is used to evaluate and rank order strengths of bimolecular interactions. The smaller the K_{D} value, the greater the binding affinity of the ligand for its target. The binding affinity is influenced by non-covalent intermolecular interactions such as hydrogen bonding, electrostatic interactions, hydrophobic and Van der Waals forces between the two molecules. A “standard assay” refers to an assay that is known in the art or could be routinely selected. Some standard assays of measuring binding affinity include ELISA, gel-shift assays, pull-down assays, equilibrium dialysis, analytical ultracentrifugation, cytometry, surface plasmon resonance (SPR), isothermal titration calorimetry and spectroscopic assays. The standard assays provide a “read-out” such as a number provided through an electronic media or printout that relates to the degree of, for example, a binding interaction directly or through a calibration. The read-out can also be as a color change that can be observed or measured, optionally using a microscope. The read-out can also be presented as a plotted data, such as a UV-Vis emission, fluorescence or absorbance.

Some embodiments include a pharmaceutical composition. As described in detail below, the pharmaceutical compositions can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, agents can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and 35 U.S. Pat. No. 3,270,960.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

Some embodiments include methods for modulating a function of microglial cells. In some embodiments the microglial cells are modulated in vitro such as in a cell culture. In other embodiments the cells are modulated in vivo, wherein the cells are in a subject. In yet other aspects the cells are modulated ex vivo, such as from a biopsy or sample from a subject.

Microglial cells function as the primary immune cells of the central nervous system (CNS), and are similar to peripheral macrophages. Once activated, for example as a response to a pathogen or injury, they function as the major inflammatory cell type in the brain. The activated cells can function to rapidly change morphology, proliferate and migrate to the site of infection/injury where through phagocytosis they destroy pathogens as well as remove damaged cells. As part of their response function microglial cells can also secrete cytokines and chemokines, as well as prostaglandins, NO and reactive oxygen species. By releasing cytokines such as CCl2 microglial cells are also important for recruiting leucocytes into the CNS. Microglia function also to interact with infiltrating T lymphocytes and, thus, mediate the immune response in the brain. As part of their function, they have the capacity to stimulate proliferation of both TH1- and TH2-CD4 positive T cells. Additionally, they function as an aid in the resolution of the inflammatory response, through the production of anti-inflammatory cytokines such as Il-10.

As used herein “phagocytosis” refers to process by which certain cells (e.g., phagocytes) ingest or engulf other cells, cell fragments, a microorganism or foreign particles. For example, by the local infolding of the cell's membrane and protrusion of its cytoplasm around the fold until the material has been surrounded and engulfed by closure of the membrane and formation of a vacuole. This a characteristic of some types of immune cells.

In some embodiments, the methods comprise administering to a patient a therapeutic dose or dosage of compositions that are identified as a possible drug in the disclosed assay. As used herein, the term “therapeutic dose” or “therapeutically effective amount” means that amount necessary, at least partly, to attain the desired effect, or to delay the onset of, inhibit the progression of, or halt altogether, the onset or progression of the particular disease or disorder being treated. This includes both therapeutic and prophylactic treatments. Such amounts will depend, of course, on the particular condition being treated, the severity of the condition and individual patient parameters including age, physical condition, size, weight and concurrent treatment. These factors are well known to those of ordinary skill in the art and can be addressed with no more than routine experimentation.

As used herein, the term “administer” or “administering” refers to the placement of a composition into a subject by a method or route which results in at least partial localization of the composition at a desired site such that a desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration. The compounds can be administered at very early stages of a disease, or before early onset, or after significant progression. When applied to an individual active ingredient, administered alone, the term refers to that ingredient alone. When applied to a combination, the term refers to combined amounts of the active ingredients that result in the therapeutic effect, whether administered in combination, serially or simultaneously.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

Some embodiments include the co-administration of compounds. This can refer to the administration of two or more compounds to a subject, wherein the two or more compounds can be administered simultaneously, or at different times, as long as they work additively or synergistically. The compounds can be administered in the same formulation or in separate formulations. When administered in separate formulations, the compounds can be administered within any time of each other. For example, the compounds can be administered within 24 hours, 12 hours, 6 hours, 5 hours, 4 hours, 3 hours, 2 hours, 1 hours, 45 minutes, 30 minute, 25 minutes, 20 minutes, 15 minutes, 10 minutes, 5 minutes or less of each other. When administered in separate formulations, any compound can be administered first. Additionally, co-administration does not require the different compounds to be administered by the same route, i.e., the components of the combination can be administered to a subject by the same or different routes of administration. As such, each can be administered independently or as a common dosage form. Similarly, the term “co-testing” can refer to the testing of two or more compounds in an assay, wherein the two or more compounds can be tested simultaneously, or at different times, for example, to determine if they work additively or synergistically. When two or more compounds are co-tested, it may not be known previously known if the compounds work synergistically and the testing can be to determine if a synergistic effect exists, if two compounds are compatible, if an additive effect exists, if a negative synergistic effect exists or any other combined effects exist.

As referred to herein, the screening assay or testing can be performed in any suitable container or apparatus available to one of skill in the art for cell culturing. For example, the assay can be performed in 24-, 96-, or 384-well plates. In one embodiment, the assay is performed in a 384-well plate.

In some embodiments, the screening method or testing is a high-throughput screening. High-throughput screening (HTS) is a method for scientific experimentation that uses robotics, data processing and control software, liquid handling devices, and sensitive detectors. High-Throughput Screening or HTS allows a researcher to quickly conduct millions of biochemical, genetic or pharmacological tests. High-Throughput Screening are well known to one skilled in the art, for example, those described in U.S. Pat. Nos. 5,976,813; 6,472,144; 6,692,856; 6,824,982; and 7,091,048, and contents of each of which is herein incorporated by reference in its entirety.

HTS uses automation to run a screen of an assay against a library of candidate compounds. Typical HTS screening libraries or “decks” can contain from 100,000 to more than 2,000,000 compounds.

The key labware or testing vessel of HTS is the microtiter plate: a small container, usually disposable and made of plastic, which features a grid of small, open divots called wells. Modern microplates for HTS generally have either 384, 1536, or 3456 wells. These are all multiples of 96, reflecting the original 96 well microplate with 8×12 9 mm spaced wells. In some embodiments automation is utilized with the larger well plates, for example having 24 or 96 well plates.

To prepare for an assay, the researcher fills each well of the plate with the appropriate reagents that he or she wishes to conduct the experiment with, such as a cell. After some incubation time has passed to allow the reagent to absorb, bind to, or otherwise react (or fail to react) with the compounds in the wells, measurements are taken across all the plate's wells, either manually or by a machine. Manual measurements are often necessary when the researcher is using microscopy to (for example) seek changes that a computer could not easily determine by itself. Otherwise, a specialized automated analysis machine can run a number of experiments on the wells such as colorimetric measurements, radioactivity counting, etc. In this case, the machine outputs the result of each experiment as a grid of numeric values, with each number mapping to the value obtained from a single well. A high-capacity analysis machine can measure dozens of plates in the space of a few minutes like this, generating thousands of experimental data points very quickly.

### Some Selected Definitions

For convenience, certain terms employed herein, in the specification, examples and appended claims are collected herein. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

As used herein the term “comprising”, “comprises”, “includes” or “including” is used in reference to compositions, methods, and respective component(s) thereof, that are essential to the invention, yet open to the inclusion of unspecified elements, whether essential or not.

The singular terms “a,” “an,” and “the” include plural referents unless context clearly indicates otherwise. Similarly, the word “or” is intended to include “and” unless the context clearly indicates otherwise.

Other than in the operating examples, or where otherwise indicated, all numbers expressing quantities of ingredients or reaction conditions used herein should be understood as modified in all instances by the term “about.” The term “about” when used in connection with percentages may mean±5% (e.g., ±4%, ±3%, ±2%, or ±1%) of the value being referred to.

Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

As used herein, the term “herein” is used to refer to the whole disclosure and is not meant to be restricted to a specific section or subsection of the disclosure.

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least at least 1% as compared to a reference level, for example decrease by at least about 10%, or at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 1-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 1% as compared to a reference level, for example an increase of about 10% as compared to a reference level, or of at least about 20%, or at least about 30%, or at least about 40%, or at least about 50%, or at least about 60%, or at least about 70%, or at least about 80%, or at least about 90% or up to and including a 100% increase or any increase between 1-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means at least two standard deviation (2SD) away from a reference level. The term refers to statistical evidence that there is a difference. It is defined as the probability of making a decision to reject the null hypothesis when the null hypothesis is actually true.

A “cell line” refers to a population of largely or substantially identical cells that has typically been derived from a single ancestor cell or from a defined and/or substantially identical population of ancestor cells. The cell line may have been or may be capable of being maintained in culture for an extended period (e.g., months, years, for an unlimited period of time). It may have undergone a spontaneous or induced process of transformation conferring an unlimited culture lifespan on the cells. Cell lines include all those cell lines recognized in the art as such. It will be appreciated that cells acquire mutations and possibly epigenetic changes over time such that at least some properties of individual cells of a cell line may differ with respect to each other.

An “isolated cell” as can be used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched from. In some embodiments, the isolated population is an isolated population of reprogrammed cells which is a substantially pure population of reprogrammed cells as compared to a heterogeneous population of cells comprising reprogrammed cells and cells from which the reprogrammed cells were derived.

A “substantially pure” cell population, can refer to a particular cell population that is at least about 75%, at least about 85%, at least about 90%, or at least about 95% pure, with respect to the cells making up a total cell population.

The disclosure is further illustrated by the following examples which should not be construed as limiting. The examples are illustrative only, and are not intended to limit, in any manner, any of the aspects described herein. The following examples do not in any way limit the invention.

### Examples

### General

As shown in the figures, compounds were selected and tested in microglial and 3D AD cell culture models at a range of concentrations, and over a period of time ranging from hours to days. The culture models were chosen to elucidate different stages and biological aspects of Alzheimer's disease. The toxicity of the compounds was assessed analyzing the cultures media with the commercially available assay (CytoTox-ONE™, Promega) that measures the release of LDH. Toxic concentrations were excluded from further testing. For microglial cultures, Aβ42 and Aβ40 uptake assays were carried forward for 2 to 24 hours following 3 hours of compound pre-treatment. The experimental outcomes were determined by measuring increased internalized Aβ42 and Aβ40 levels by a commercially available ELISA assay (Wako Abeta42 ELISA kit). Alternatively, LPS-activation assays were carried forward for 3 h following 3 hours of compound pre-treatment. The reduction of toxic cytokines released in the culture media was monitored.

Experimental Information

Naïve BV2 Cells, or BV2 stably expressing wt-CD33, were seeded in 24-well plates at the density of 2.5×10E5 cells for BV2 and 4×10E5 cells for wt-CD33 clone, in proliferating media. On the following day, cells were treated with compounds, or DMSO as a control, at different concentrations in proliferating media for 3 hours. For Abeta42 and Abeta40 uptake assays, cells were washed twice with 500 μL/well of PBS and treated with compounds/DMSO in the presence of 300 nM Abeta peptide in DMEM media for 2 hours. At the end of the 2 hour incubation, 150 μL of media were collected from the 24-well plates. They were centrifuged at 4° C., 2500 rpm for 10 min, transferred to new plates and used to assess compounds toxicity with CytoTox-ONE™ (LDH) assay. The remaining cells in the 24-well plates were washed three times with 500 μL/well of cold PBS and lysed with 50 μL of RIPA buffer supplemented with Complete™ EDTA-free protease inhibitors cocktail, HALT™ phosphatase inhibitor and 1,10-Phenantroline, while rocking for 20 min at 4° C. The Cells were centrifuged at 4° C., 13,500 rpm for 15 min and the supernatant was transferred to new microcentrifuge tubes. Protein concentrations in the lysate supernatants were determined with the Pierce™ BCA protein assay kit. 2-3 μg/well of protein from the lysates was analyzed for Abeta42 uptake using the Wako Abeta42 ELISA kit. For Abeta40 uptake, Cisbio Abeta40 HTRF assay was used.

Toxic compound concentrations were excluded from the Abeta42 and Abeta40 analysis.

For microglial activation experiments, cells were treated with compounds/DMSO in the presence of 1 μg/mL LPS in proliferating media for 3 hours. The media was collected, cleared of particulate and analyzed using MSD Proinflammatory Panel 1 cytokine assay kit.

### Testing of Compound 817 MA

The protocol used for 24-hour pre-treatment testing in microglial cells was as follows.

On day zero the naïve BV2 microglial cells were seeded in proliferating media.

On day one the cells were then treated with 817MA, or DMSO as a control, in proliferating media for 24 hours, at concentrations ranging from 0 to 50 μM.

On day two the cells were then washed twice with PBS and treated with compounds/DMSO in the presence of 300 nM Abeta42 peptide in DMEM media for 2 hours. The compounds toxicity was assessed in the media collected at the end of the treatment with CytoTox-ONE™ (LDH) assay. The remaining cells were lysed with RIPA buffer supplemented with protease and phosphatase inhibitors. Protein concentrations were determined with the Pierce™ BCA protein assay kit. Normalized lysates were analyzed for Abeta42 uptake using the Wako Abeta42 ELISA kit.

### LDH Toxicity Testing

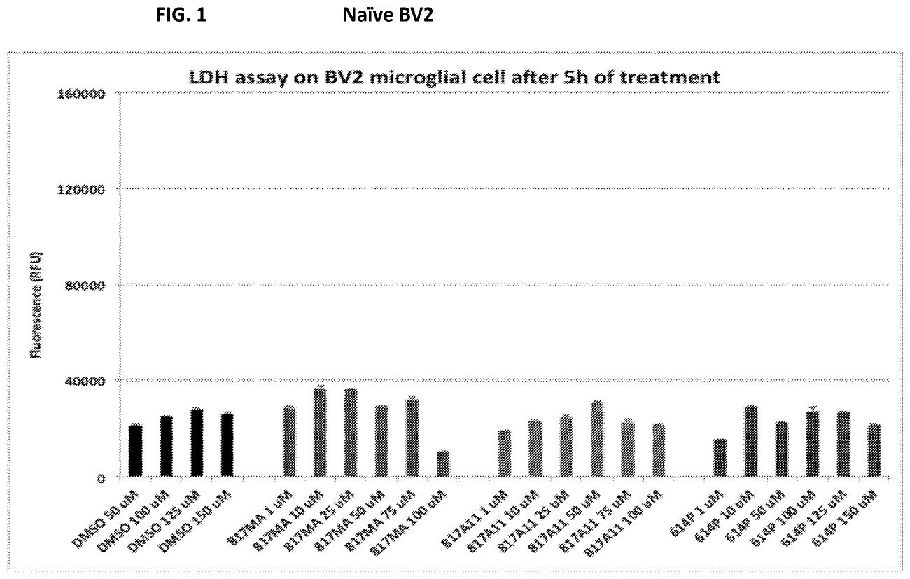

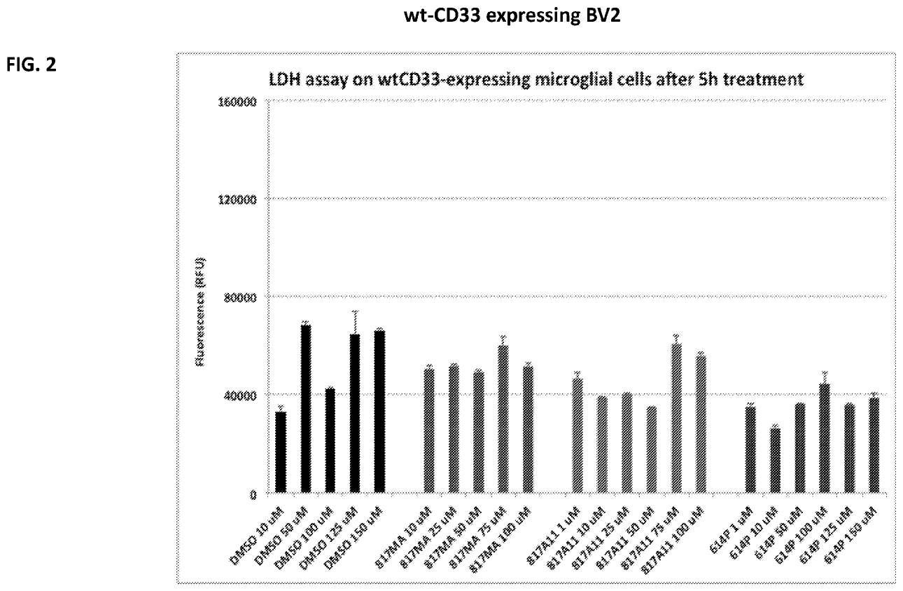

Toxicity testing results is shown with reference to FIGS. 1 and 2. Both figures show the results as a bar graph for LDH toxicity. The test compounds are DMSO, 817MA, 817A11, and 614P. FIG. 1 shows the toxicity testing results on naïve microglial cells and FIG. 2 shows the results on wtCD33-expressing microglial cells. In both cases no statistically significant toxicity is seen for the test compounds after a five-hour treatment of the cells with the compounds.

### Abeta Uptake Assay

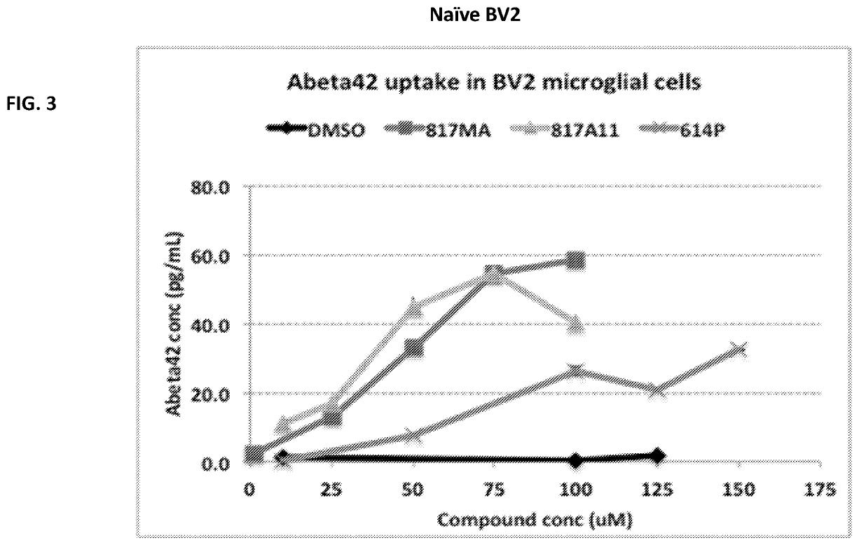

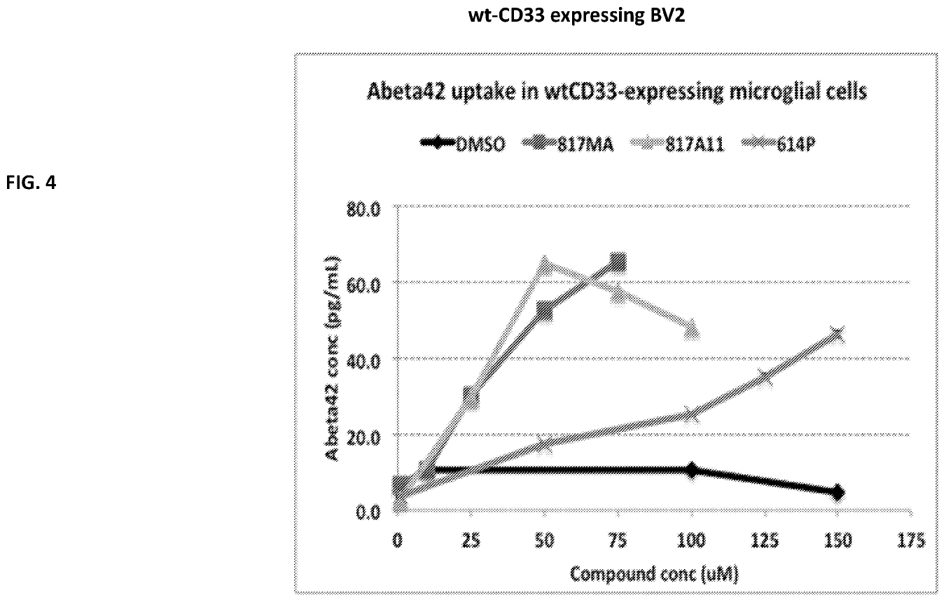

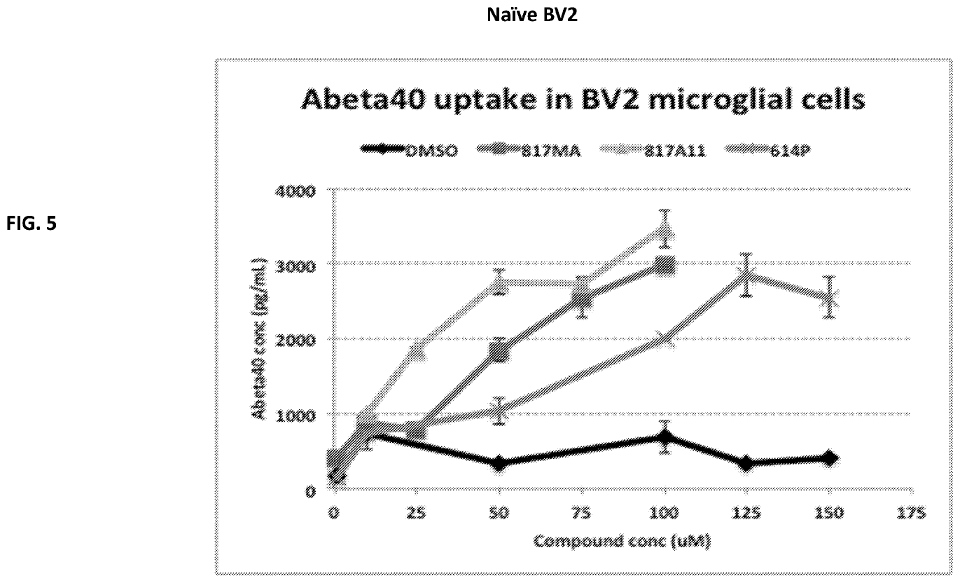

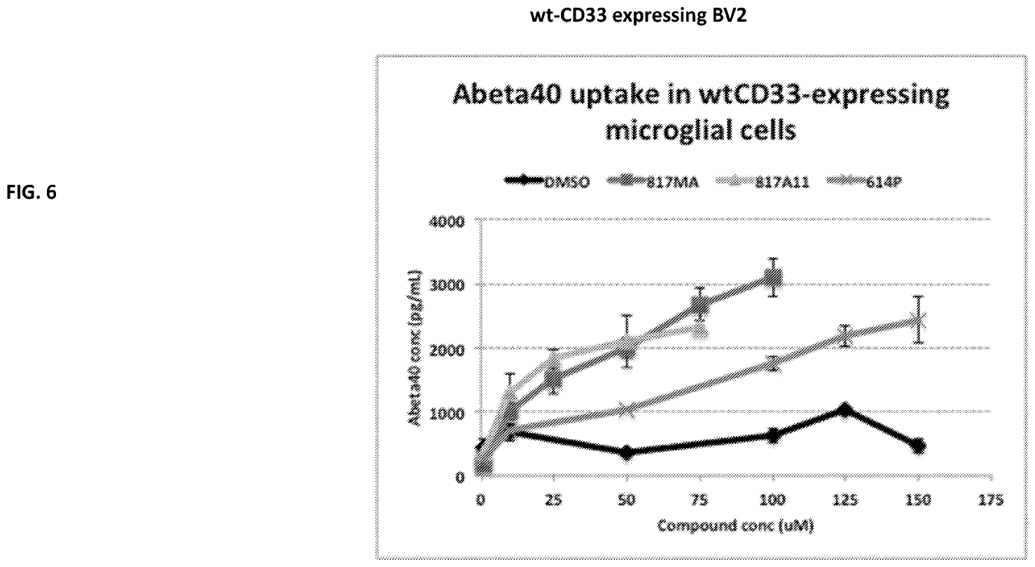

Abeta uptake assays with test compounds DMSO, 817MA, 817A11 and 614P is shown with reference to the results displayed by FIG. 3-6. The effect on the uptake of Abeta40 and Abeta42 into naïve BV2 and wt-CD33 expressing BV2 cells as a function of concentration is assayed. FIG. 3 shows that 817MA and 817MA have a lower EC₅₀ for the uptake of Abeta40 and Abeta42 in all the cell types tested as compared to test compound 614P. The assay also distinguishes between 817MA and 817A11. For example, FIGS. 3 and 4 show that 817MA and 817A11 treated cells have similar Abeta42 uptake in naïve BV2 and wt-CD33 expressing BV2 cells, while FIGS. 5 and 6 show that 817A11 treated cells have a lower EC₅₀ Abeta40 uptake than 817MA treated cells in naïve BV2 and wt-CD33 expressing BV2 cells.

### Detection and Reduction of Cytokines

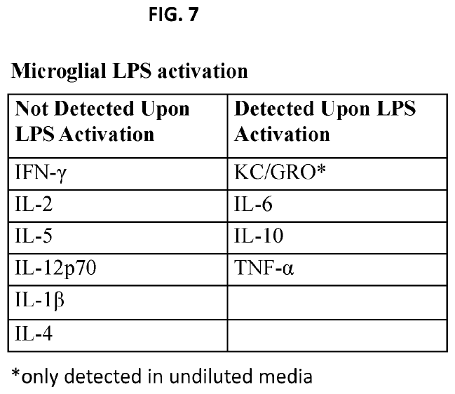

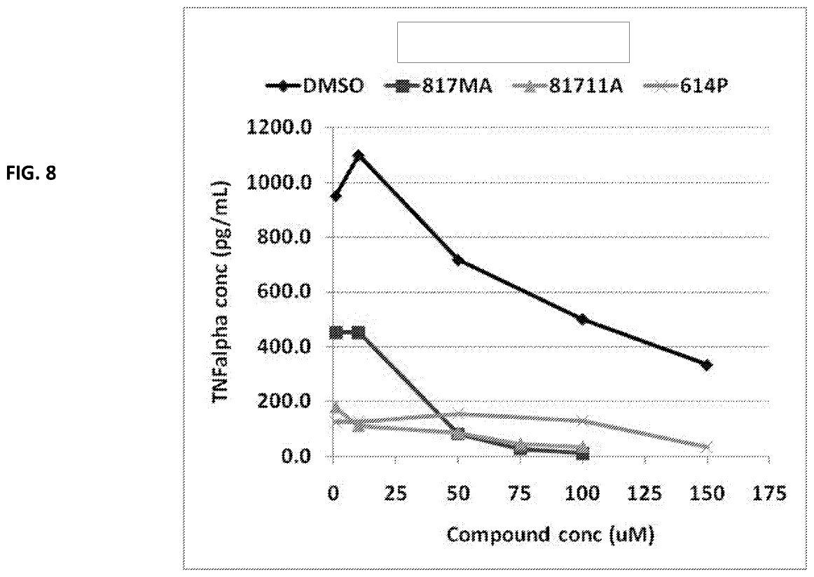

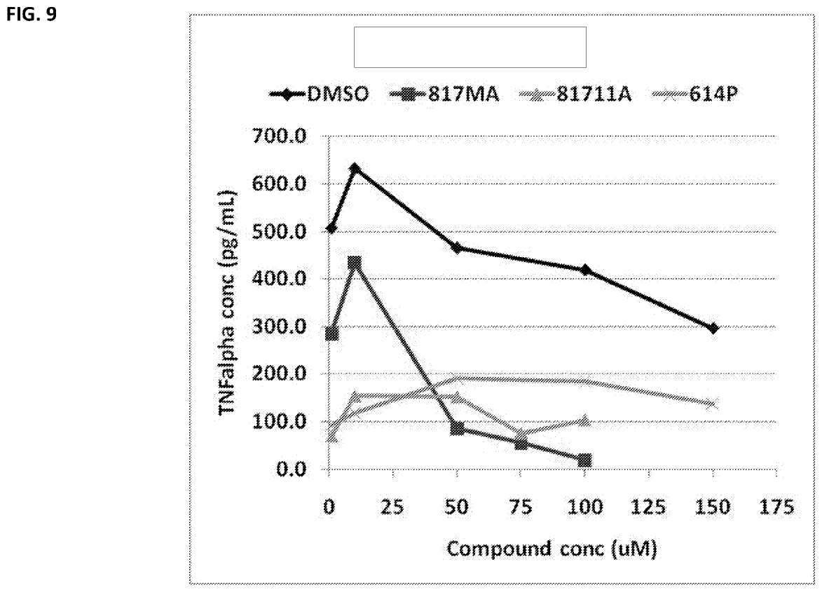

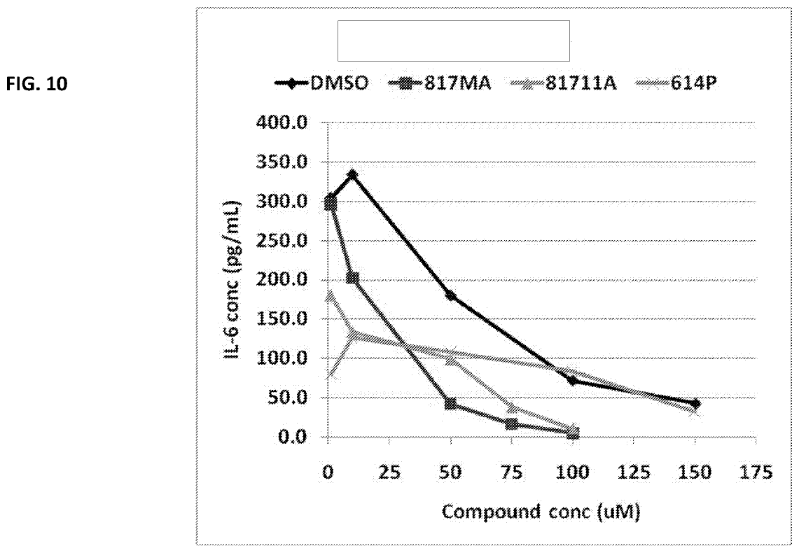

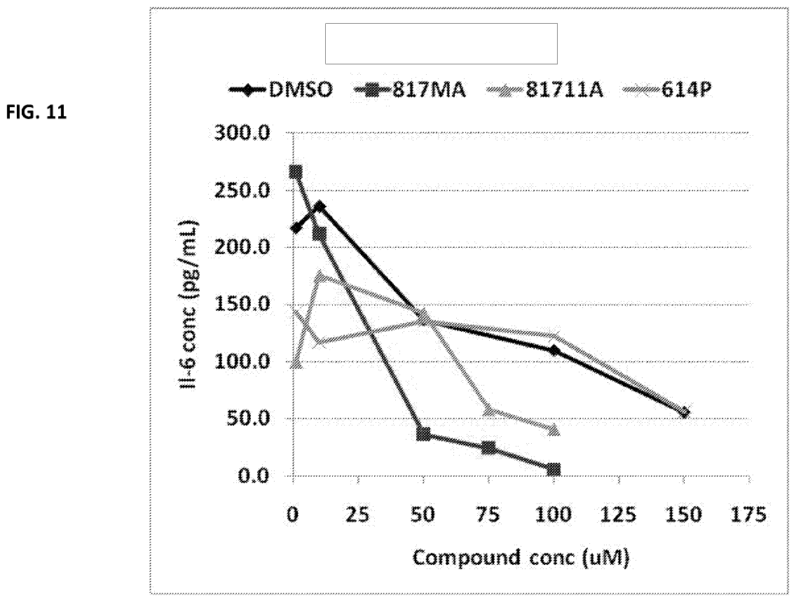

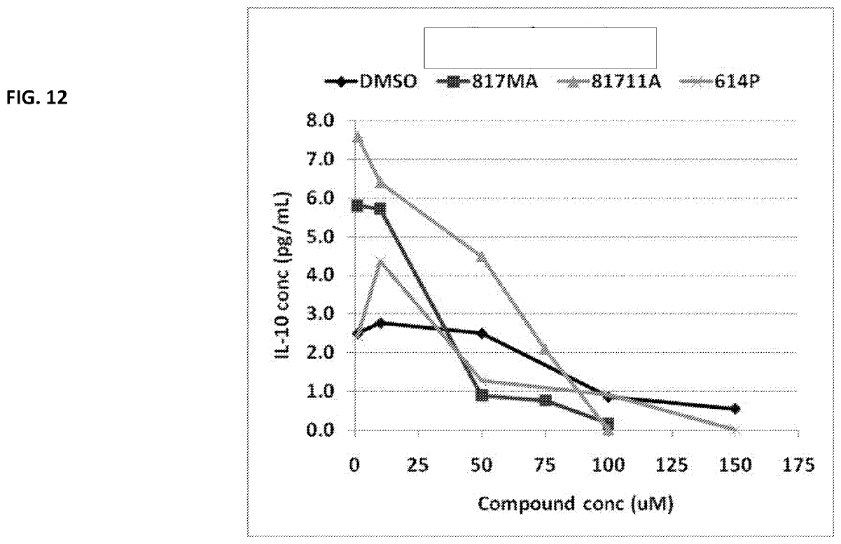

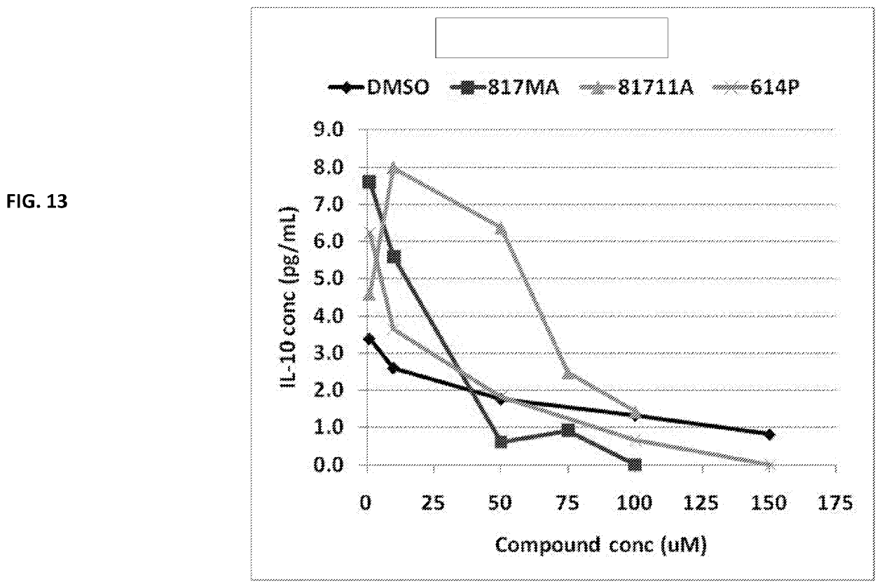

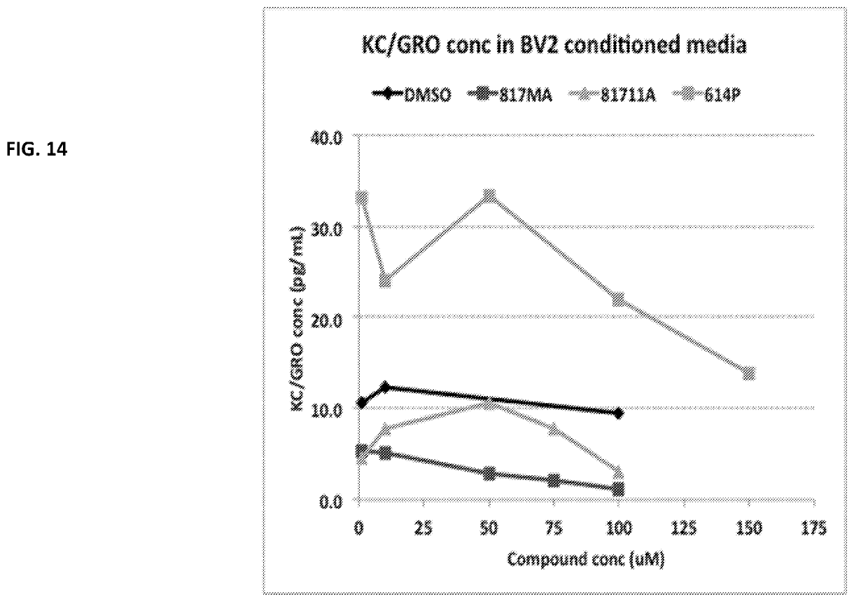

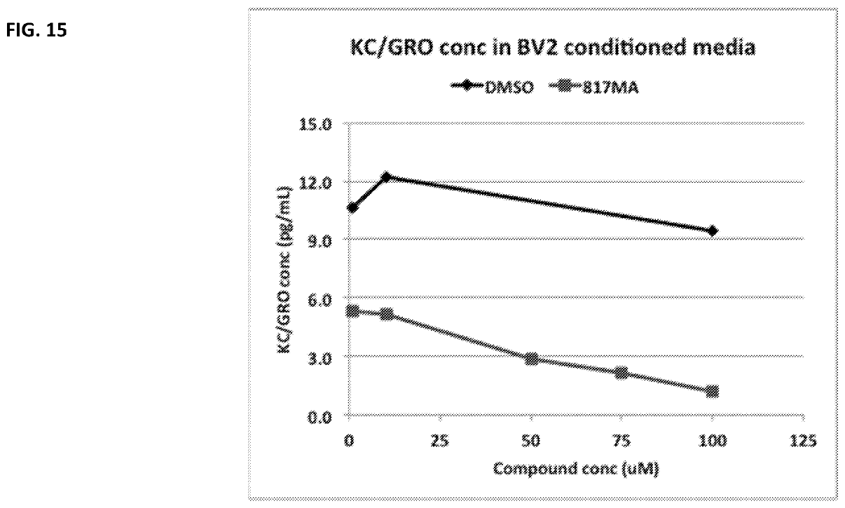

FIG. 7 tabulates the results from an LPS activation testing of microglial cells, showing that cytokines KC/GRO, IL-6, IL-10 and TNF-α were detectable upon LPS activation, although KC/GRO was only detectable in undiluted media. The 10 cytokines were simultaneously analyzed in microglial conditioned media. FIG. 8-15 show the results for an assay to test the effect of test compounds DMSO, 817MA, 817A11 and 614P on cytokines KC/GRO, IL-6, IL-10 and TNF-α. The assays show that BV2 cells treated with 817MA show reduced concentrations of IL-6, IL-10 and TNF-α. The two sequential assays determine which if any cytokines are detected upon LPS activation, followed by determination of the reduction in detectable cytokines upon treatment/exposure to a test compound.

### Testing in AD 3D Cell Culture

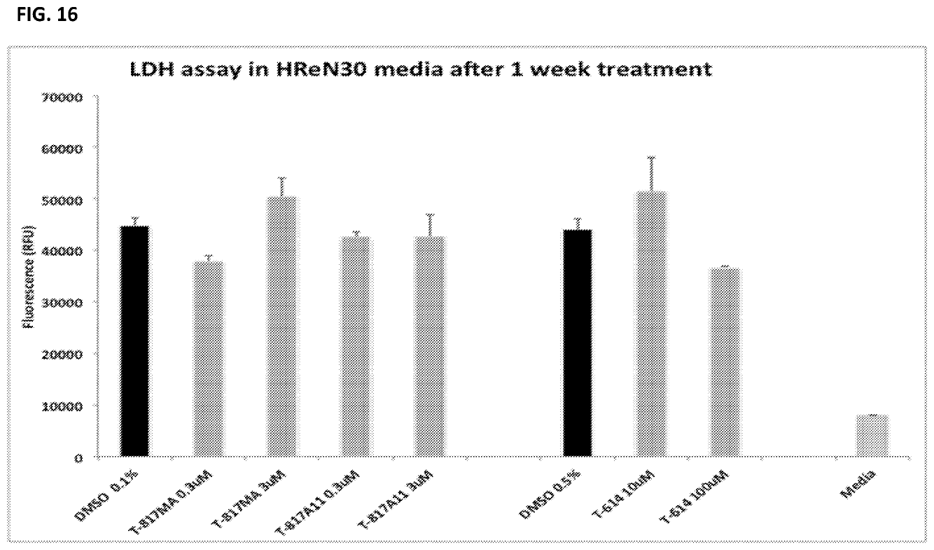

FIG. 16 shows the results in a bar graph form for data from a first test in AD 3D ReN cell culture system. The test is and LDH assay in HReN30-mGAP30 media after one-week treatment with test compounds DMSO, T-817MA (817MA), T-817A11(817A11) and T-614 (614P). n is 3 or 4 for each cell line.

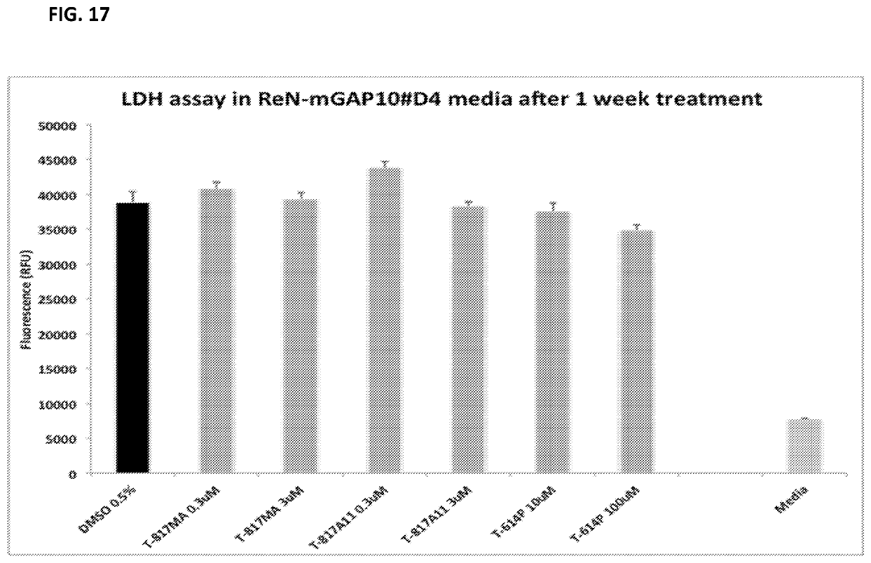

FIG. 17 shows the results in a bar graph form for data from a second test in AD 3D ReN cell culture system. The test is an LDH assay in HReN30-mGAP10# D4 media after one-week treatment with test compounds DMSO, T-817MA (817MA), T-817A11(817A11) and T-614 (614P). n is 3 or 4 for each cell line.

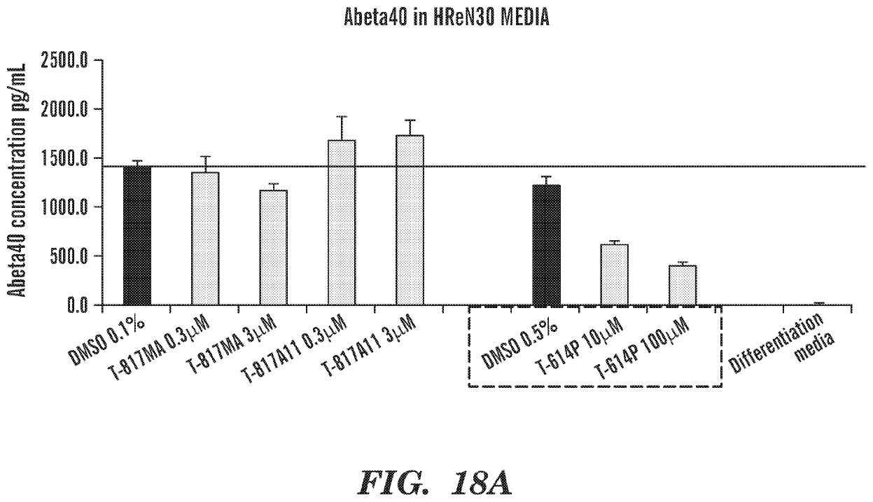

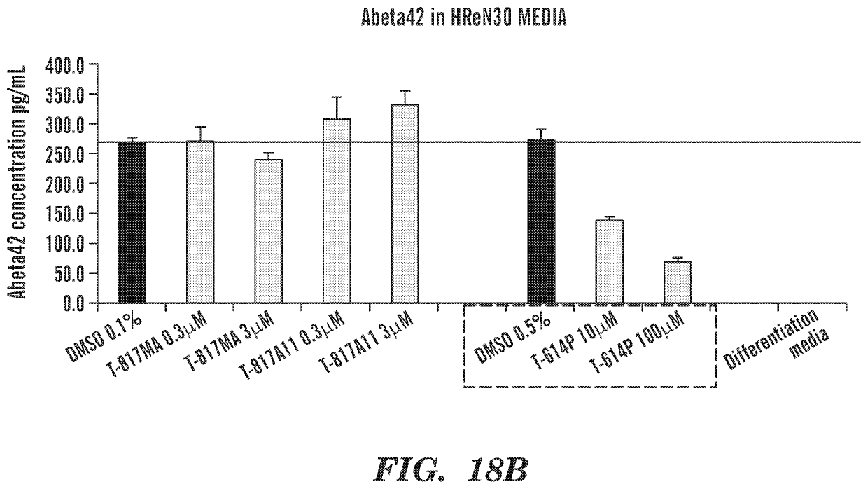

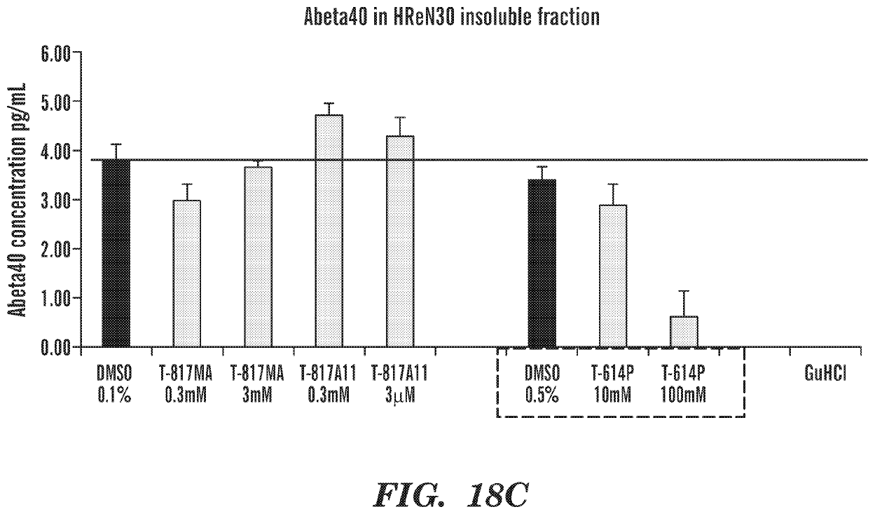

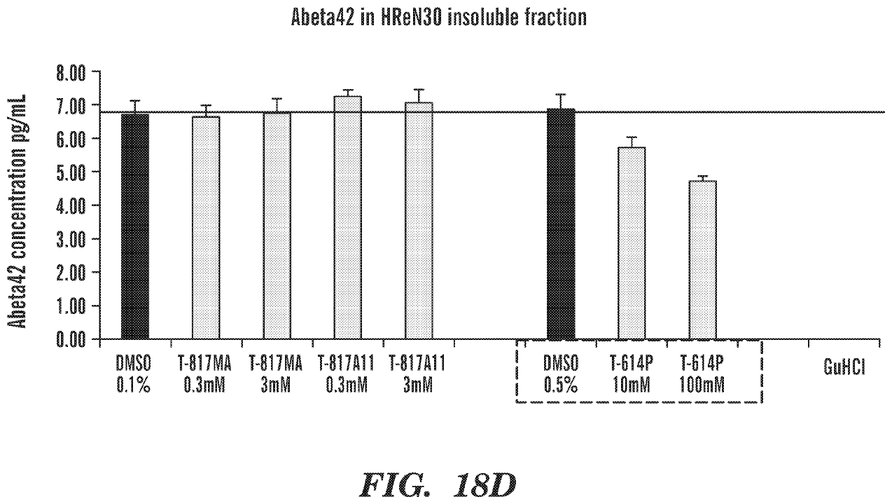

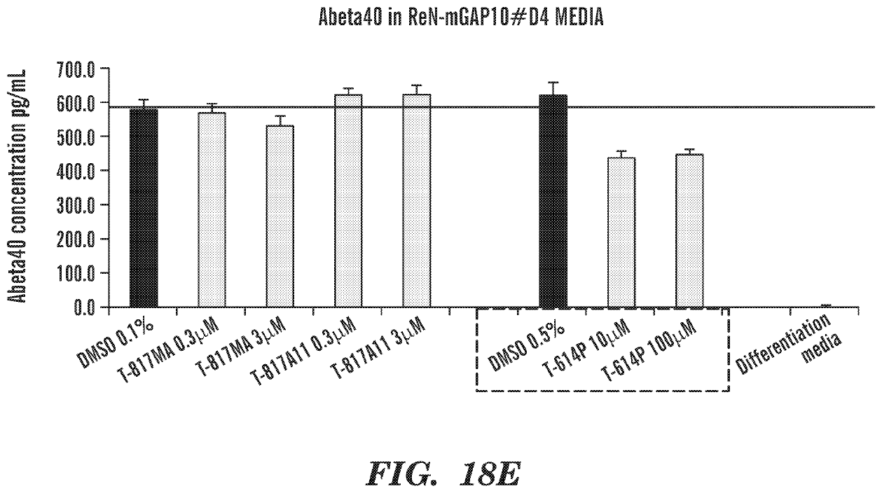

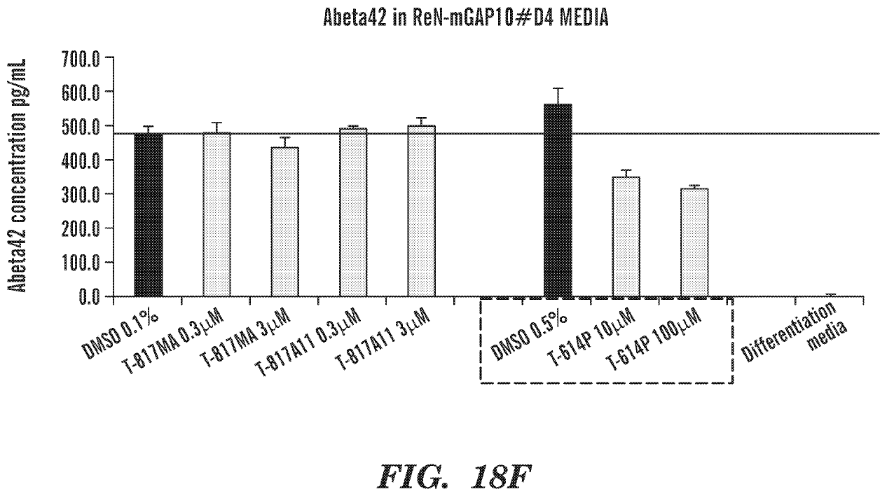

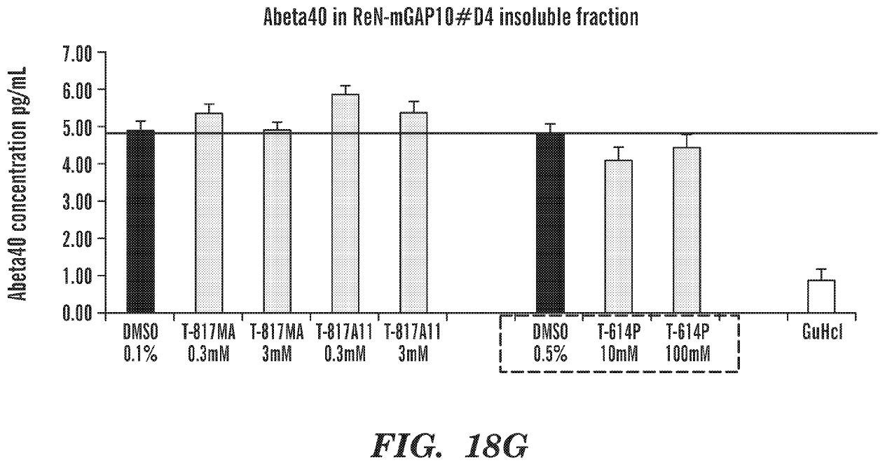

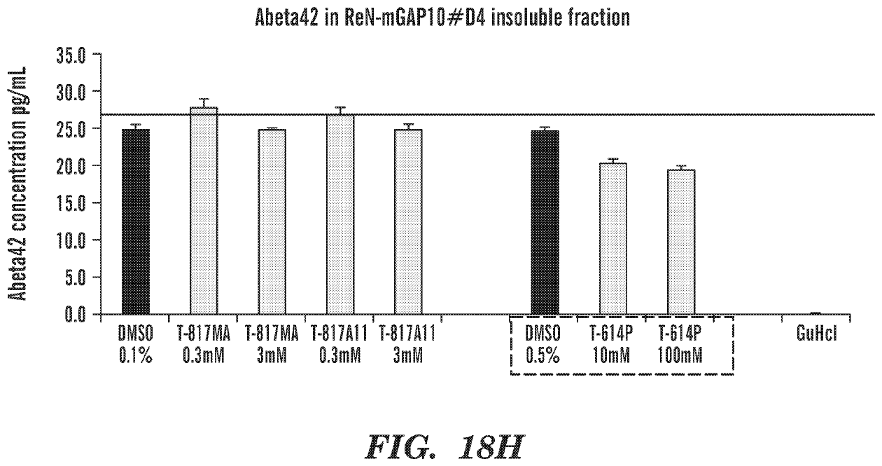

FIG. 18A-18H shows a series of bar graphs of data for soluble (media) (horizontal axis) and insoluble Abeta levels (vertical axis) after drug treatments. The test compounds are T-817MA (817MA), T-817A11(817A11) and T-614 (614P). From left to right in each of these graphs each bar is for: DMSO (0.1%), T-817MA (0.3 μM), T-817MA (3 μm), T-817A11 (0.3 μM), T-817A11 (3 μM), DMSO (0.5%), T-614P (10 μM), T-614P (100 μM) and GuHCl/differentiation media. FIG. 18A to FIG. 18D are HReN-mGAP30 experiments. FIG. 18A shows Abeta40 in HReN30 (HReN-mGAP30) Media. FIG. 18B shows Abeta42 in HReN30 (HReN-mGAP30) Media. FIG. 18C shows Abeta40 in HReN30 (HReN-mGAP30) insoluble fraction. FIG. 18D shows Abeta42 in HReN30 (HReN-mGAP30) insoluble fraction. FIG. 18E to FIG. 18H are ReN-mGAP # D4 experiments. FIG. 18E shows Abeta40 in ReN-mGAP10# D4 (ReN-mGAP # D4) Media. FIG. 18F shows Abeta42 in ReN-mGAP10# D4 (ReN-mGAP # D4) Media. FIG. 18G shows Abeta40 in ReN-mGAP10# D4 (ReN-mGAP # D4) insoluble fraction. FIG. 18H shows Abeta42 in ReN-mGAP10# D4 (ReN-mGAP # D4) insoluble fraction.

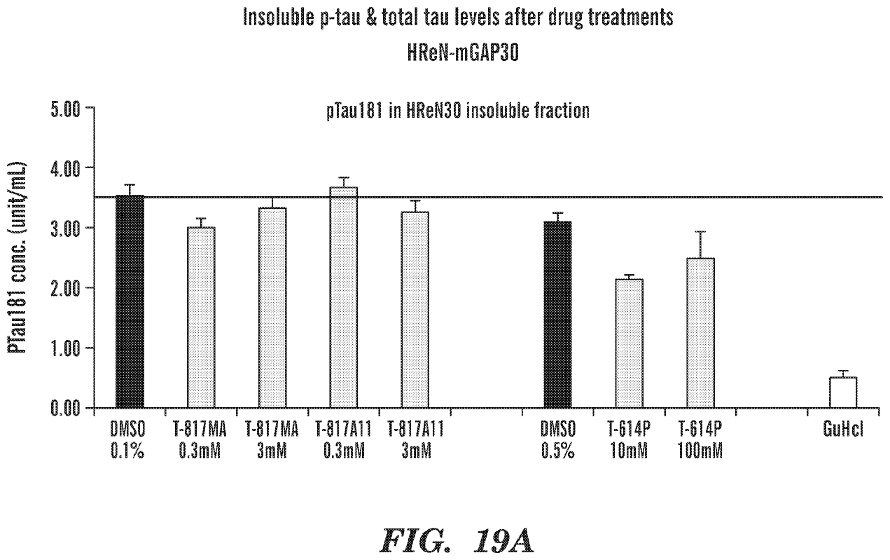

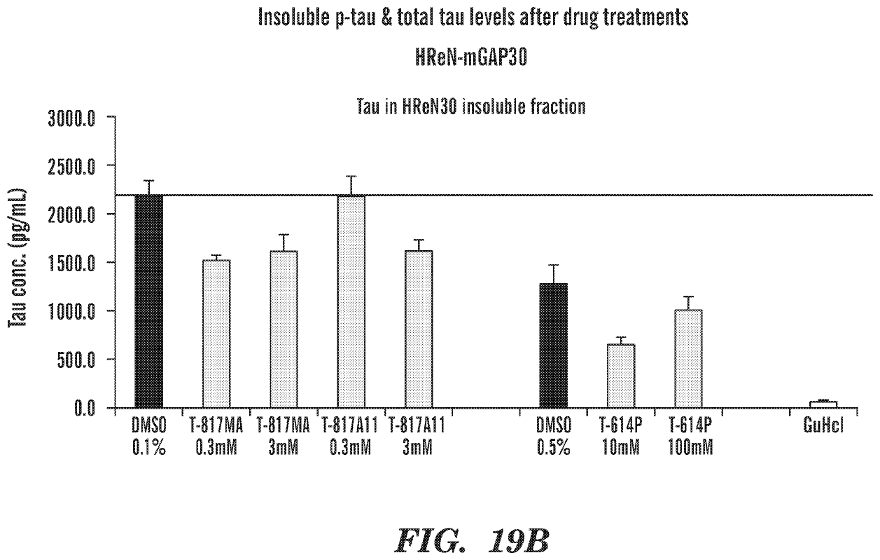

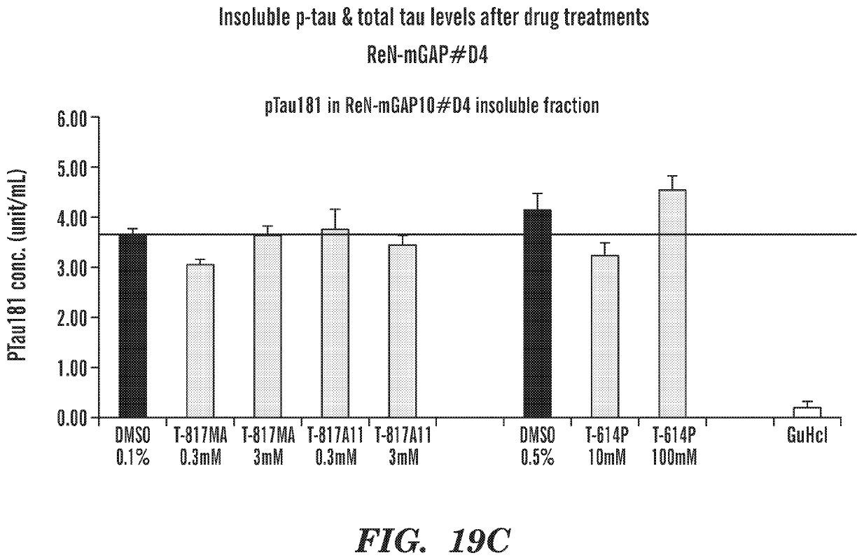

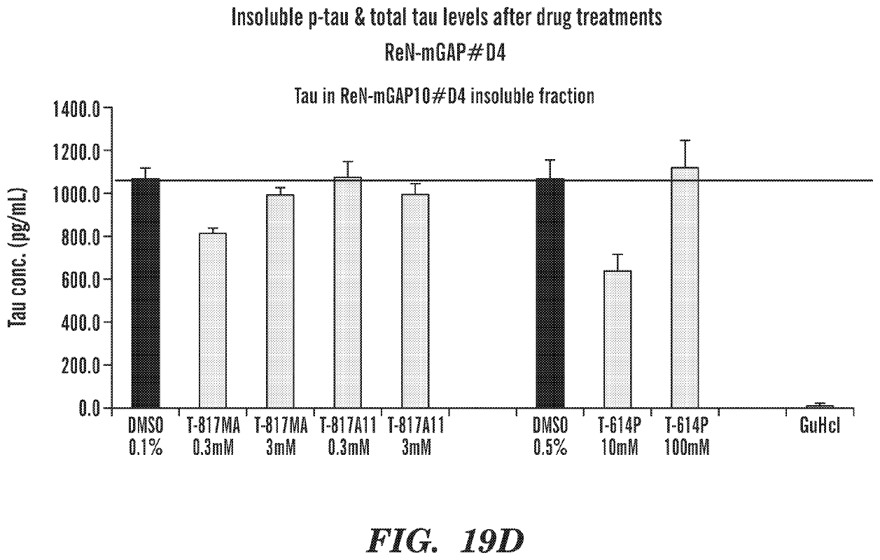

FIG. 19A-19D shows a series of bar graphs of data for insoluble p-tau and total p-tau levels after drug treatments. The test compounds are DMSO, T-817MA (817MA), T-817A11(817A11) and T-614 (614P). From left to right in each of these graphs the data is for: DMSO (0.1%), T-817MA (0.3 μM), T-817MA (3 μm), T-817A11 (0.3 μM), T-817A11 (3 μM), DMSO (0.5%), T-614P (10 μM), T-614P (100 μM) and GuHCl. FIG. 19A and FIG. 19B are HReN-mGAP30 tests. FIG. 19A shows pTau181 concentration (unit/mL) in HReN30 insoluble fraction. FIG. 19B shows pTau181 concentration (pg/mL) in HReN30 insoluble fraction. FIG. 19C and FIG. 19D are ReN-mGAP # D4 tests. FIG. 19C shows pTau181 concentration (unit/mL) in ReN-mGAP10# D4 insoluble fraction. FIG. 19D shows pTau181 concentration (pg/mL) in ReN-mGAP10# D4 insoluble fraction.

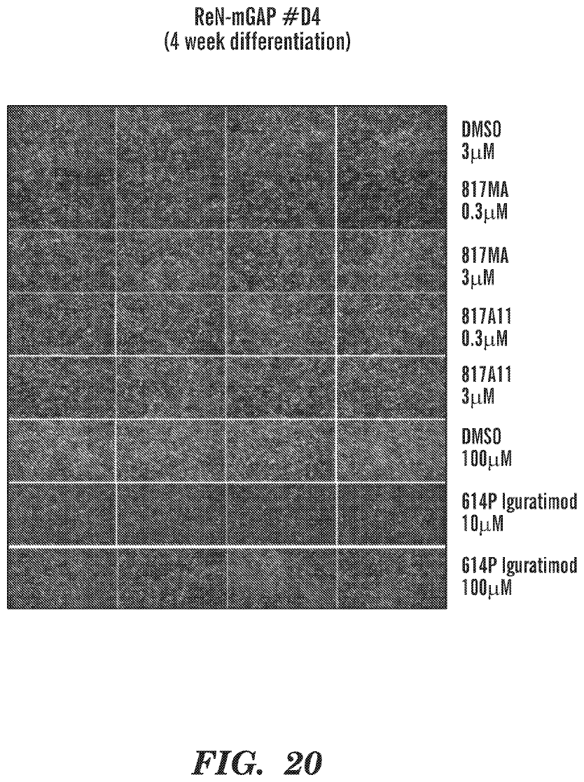

FIG. 20 shows a series of images in ReN-mGAP # D4 (4-week differentiation). The horizontal rows from top to bottom show images for DMSO 3 μM, 817MA 0.3 μM, 817MA 3 μM, 817A11 0.3 μM, 817A11 3 μM, DMSO 100 μM, 614P iguratimod 10 μM and 614P iguratimod 100 μM.

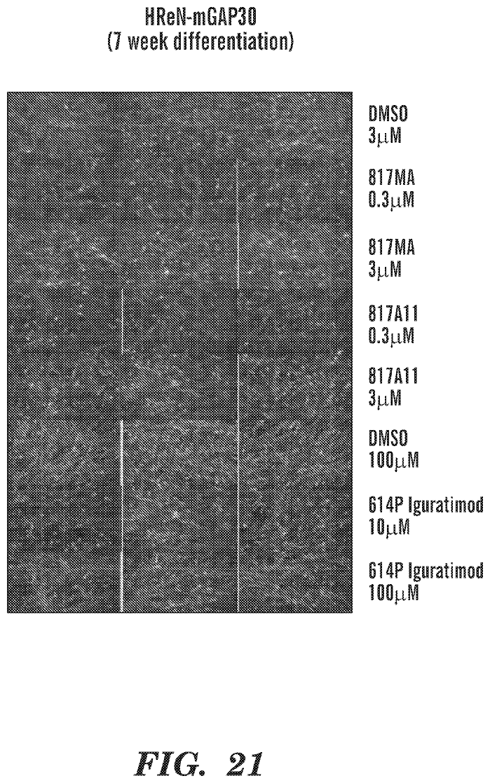

FIG. 21 shows a series of images in HReN-mGAP30 (7-week differentiation). The horizontal rows from top to bottom show images for DMSO 3 μM, 817MA 0.3 μM, 817MA 3 μM, 817A11 0.3 μM, 817A11 3 μM, DMSO 100 μM, 614P iguratimod 10 μM and 614P iguratimod 100 μM.

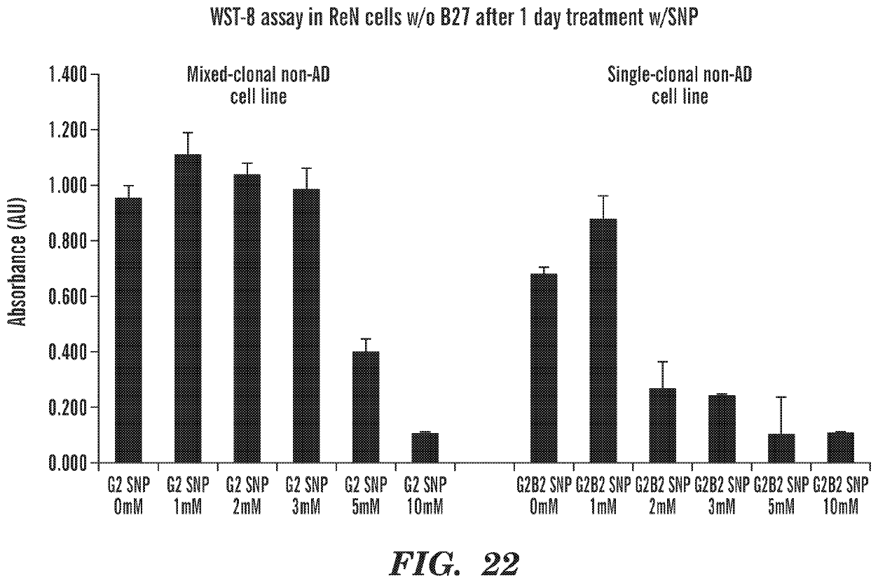

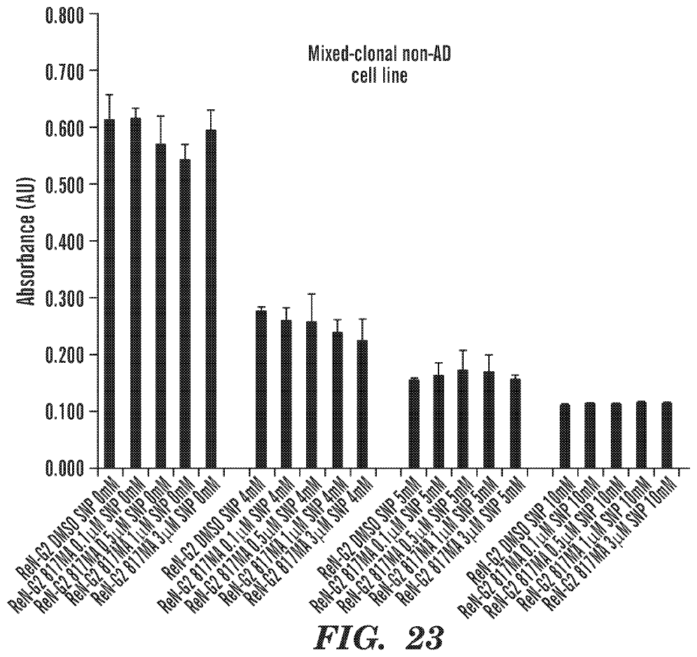

FIG. 22 shows a bar graph of data for sodium nitroprusside (SNP) toxicity studies in an AD 3D ReN cell culture system. The data is of WST-8 assay in ReN cells without B27 after one-day treatment with SNP. The 6 bars on the left are data of mixed-clonal non-AD cell line. The 6 bars on the right are data of single-clonal non-AD cell line. n=3 or 4 for each cell line.

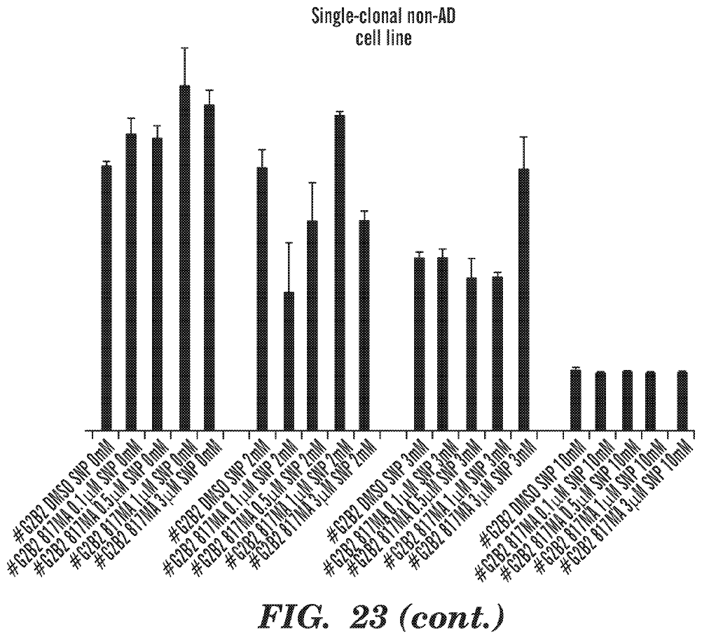

FIG. 23 shows a bar graph of data for a WST-8 assay in ReN cells without B27 after four days of 817MA and one day of SNP treatment. The 20 bars on the left are data of mixed-clonal non-AD cell line. The 20 bars on the right are data of single-clonal non-AD cell line. n=3 or 4 for each cell line. The concentrations for the treatments from left to right for the 20 bars on the left are as follows: ReN-G2 DMSO SNP 0 mM; ReN-G2 817MA 0.1 μM SNP 0 mM; ReN-G2 817MA 0.5 μM SNP 0 mM; ReN-G2 817MA 1 μM SNP 0 mM; ReN-G2 817MA 3 μM SNP 0 mM; ReN-G2 DMSO SNP 4 mM; ReN-G2 817MA 0.1 μM SNP 4 mM; ReN-G2 817MA 0.5 μM SNP 4 mM; ReN-G2 817MA 1 μM SNP 4 mM; ReN-G2 817MA 3 μM SNP 4 mM; ReN-G2 DMSO SNP 5 mM; ReN-G2 817MA 0.1 μM SNP 5 mM; ReN-G2 817MA 0.5 μM SNP 5 mM; ReN-G2 817MA 1 μM SNP 5 mM; ReN-G2 817MA 3 μM SNP 5 mM; ReN-G2 DMSO SNP 10 mM; ReN-G2 817MA 0.1 μM SNP 10 mM; ReN-G2 817MA 0.5 μM SNP 10 mM; ReN-G2 817MA 1 μM SNP 10 mM; and ReN-G2 817MA 3 μM SNP 10 mM. The concentrations for the treatments from left to right for the 20 bars on the right are as follows: # G2B2 DMSO SNP 0 mM; # G2B2 817MA 0.1 μM SNP 0 mM; # G2B2 817MA 0.5 μM SNP 0 mM; # G2B2 817MA 1 μM SNP 0 mM; # G2B2 817MA 3 μM SNP 0 mM; # G2B2 DMSO SNP 2 mM; # G2B2 817MA 0.1 μM SNP 2 mM; # G2B2 817MA 0.5 μM SNP 2 mM; # G2B2 817MA 1 μM SNP 2 mM; # G2B2 817MA 3 μM SNP 2 mM; # G2B2 DMSO SNP 3 mM; # G2B2 817MA 0.1 μM SNP 3 mM; # G2B2 817MA 0.5 μM SNP 3 mM; # G2B2 817MA 1 μM SNP 3 mM; # G2B2 817MA 3 μM SNP 3 mM; # G2B2 DMSO SNP 10 mM; # G2B2 817MA 0.1 μM SNP 10 mM; # G2B2 817MA 0.5 μM SNP 10 mM; # G2B2 817MA 1 μM SNP 10 mM; and # G2B2 817MA 3 μM SNP 10 mM.

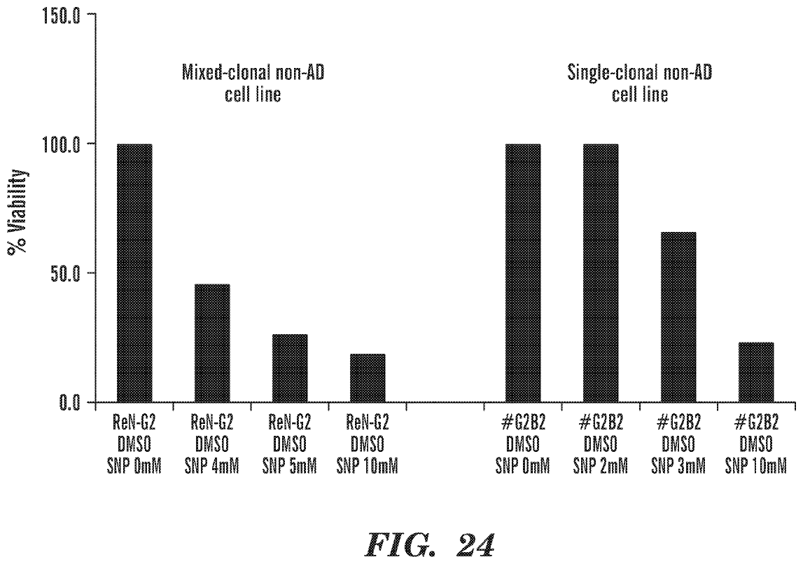

FIG. 24 shows a bar graph of data for a WST-8 assay (% viability) in ReN cells without B27 after four days of 817MA and one day of SNP treatment. The 4 bars on the left are data of mixed-clonal non-AD cell line ReN-G2 data. The 4 bars on the right are data of single-clonal non-AD cell line # G2B2 data. n=3 or 4 for each cell line

### Optimization of Drug Candidate

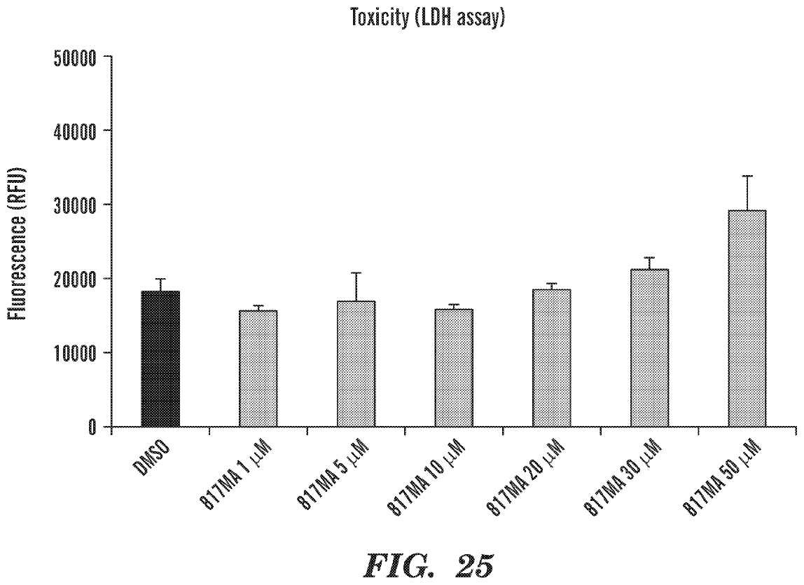

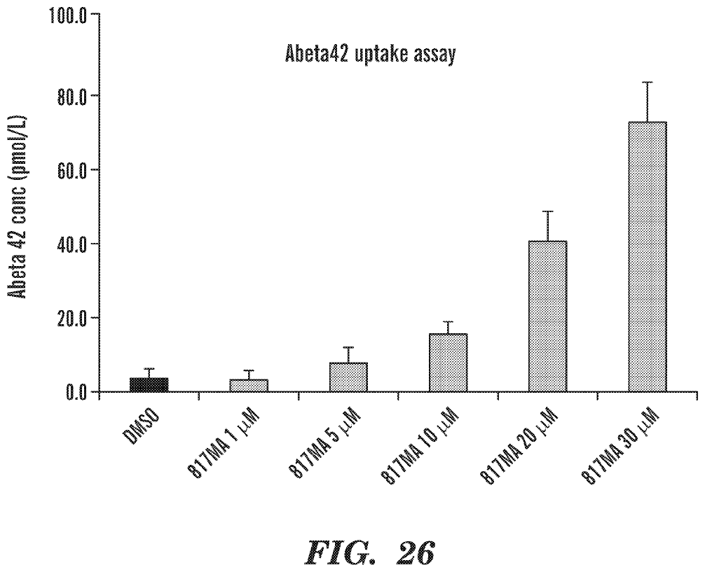

FIG. 25 shows that 817MA has low or no toxicity below about 50 μM, for example at about 30 μM no statistically significant toxicity is seen as compared to DMSO. FIG. 26 shows that above about 1 μM Abeta42 uptake is greater than the DMSO control (e.g., above about 5, above about 10, above about 20 μM) in microglial cells. The EC₅₀ is about 20 μM following 24 hrs of 817MA treatment. This example shows how optimization of drug concentration can be achieved by minimizing toxicity and maximizing Abeta uptake.

### SNP Assay in Brain 3D AD Model

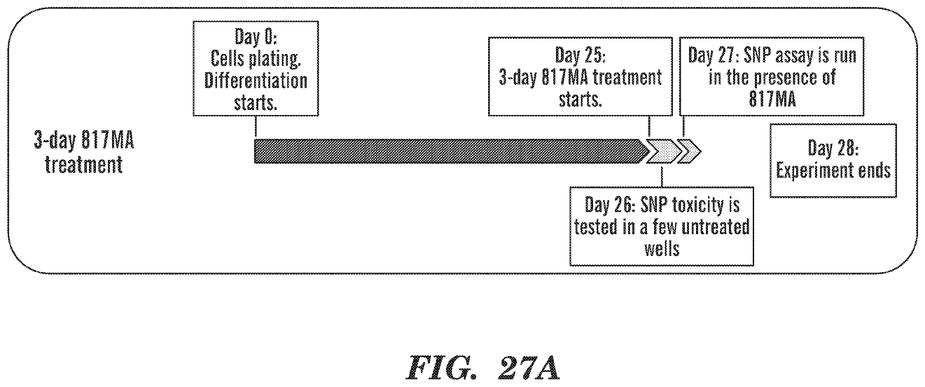

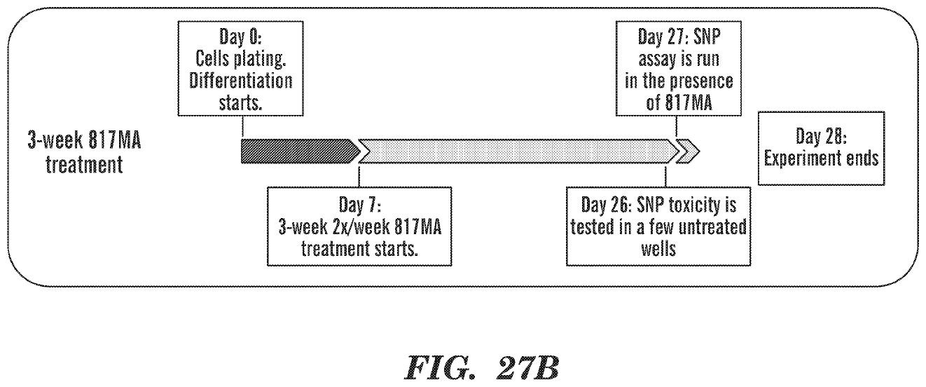

FIGS. 27A and 27B show exemplary time line diagrams for 817MA treatments. FIG. 27 A shows a 3-day 817MA treatment which includes: (day 0) cells plating, differentiation starts; (day 25) 3-day 817MA treatment starts; (day 26) SNP toxicity is tested in a few untreated wells; (day 27) SNP assay is run in the presence of 817MA; and (day 28) experiment ends. FIG. 27 B shows a 3-week 817MA treatment which includes: (day 0) cells plating, differentiation starts; (day 7) 3-week 2×/week 817MA treatment starts; (day 26) SNP toxicity is tested in a few untreated wells; (day 27) SNP assay is run in the presence of 817MA; and (day 28) experiment ends. Non-AD cell lines used were mixed clonal ReN-G2. AD cell lines used were mixed clonal HReN30; single clonal mGAP # D4 and mAP # E6F4. The read-out assay was WST-8

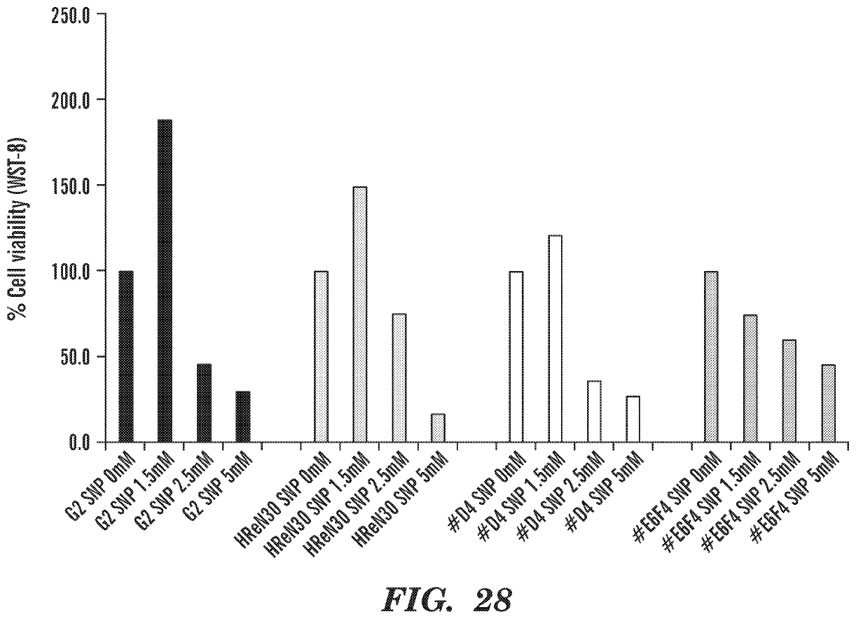

FIG. 28 shows a bar graph of data for cell viability to SNP concentration at day 26. Left to right are data for non-AD, AD mixed clonal, AD single clonal and AD single clonal.

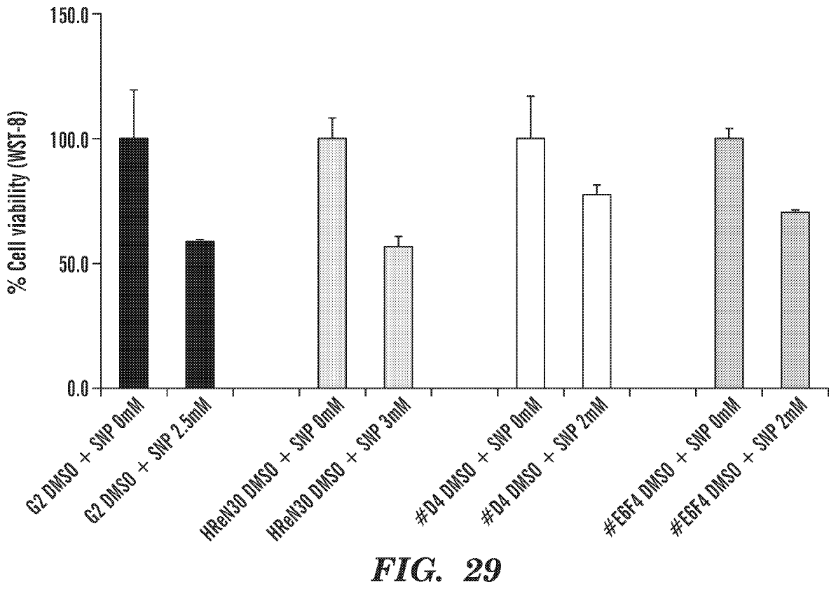

FIG. 29 shows a bar graph of data for cell viability at selected concentrations. Left to right are data for non-AD, AD mixed clonal, AD single clonal and AD single clonal. n=3 or 4 for each cell line.

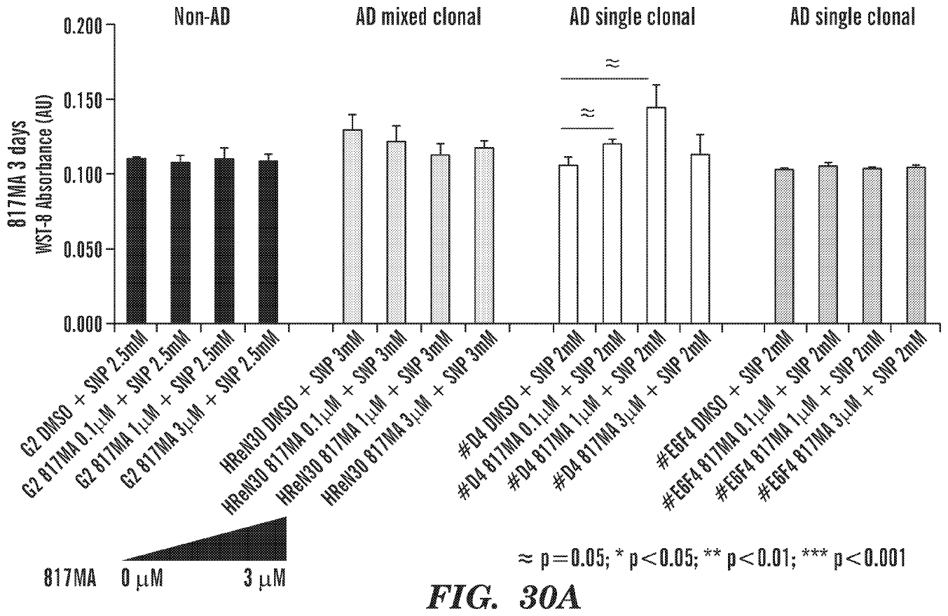

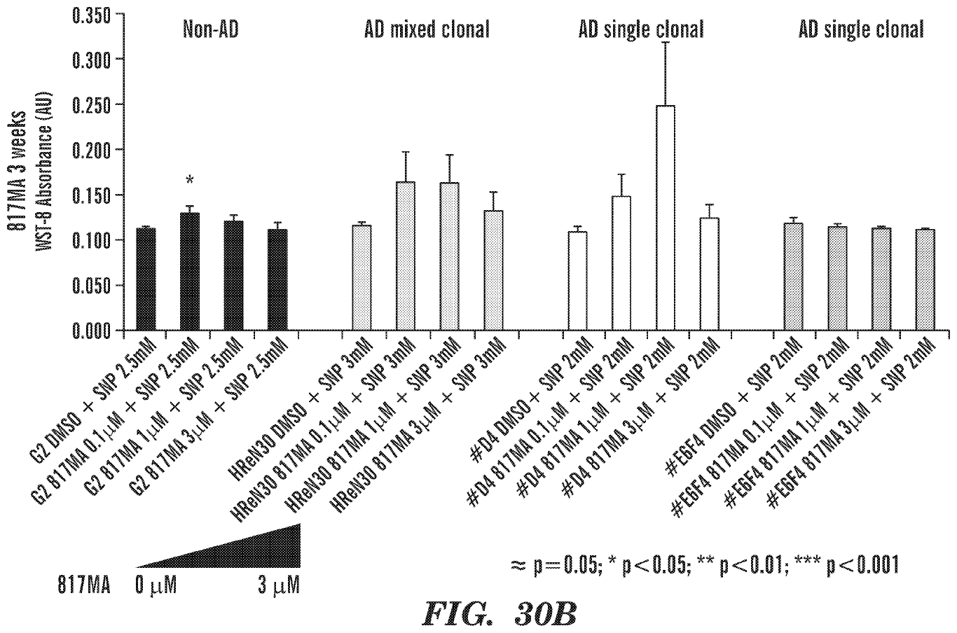

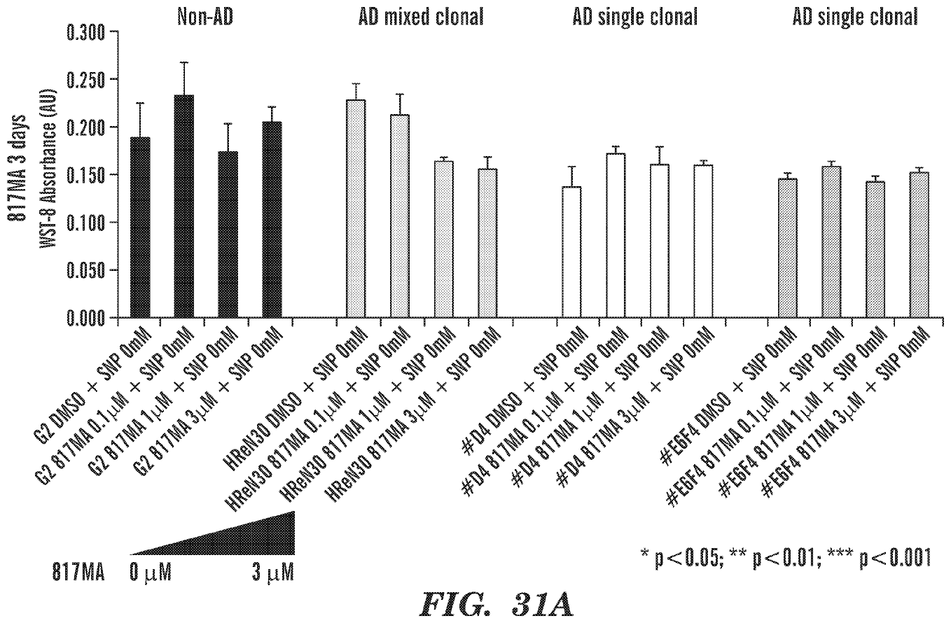

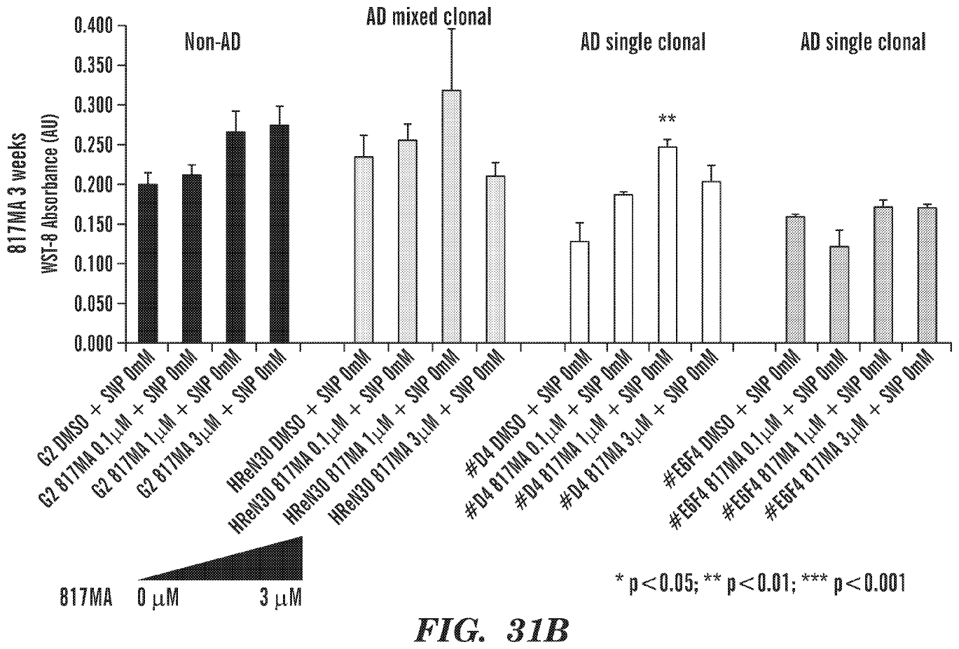

FIGS. 30A and 30B show two bar graphs showing data for the 817MA effect on SNP-induced toxicity. FIG. 31A, shows data for 817MA 3 days. FIG. 31B, shows data for 817MA 3 weeks. Left to right, in groups of four bars, are data for non-AD, AD mixed clonal, AD single clonal and AD single clonal. From left to right each bar is for: G2 DMSO+SNP 2.5 mM; G2 817MA 0.1 μM SNP 2.5 mM; G2 817MA 1 μM SNP 2.5 mM; G2 817MA 3 μM+SNP 2.5 mM; HReN30 DMSO+SNP 3 mM; HReN30 817MA 0.1 μM SNP 3 mM; HReN30 817MA 1 μM SNP 3 mM; HReN30 817MA 3 μM+SNP 3 mM; # D4 DMSO+SNP 2 mM; # D4 817MA 0.1 μM SNP 2 mM; # D4 817MA 1 μM SNP 2 mM; # D4 817MA 3 μM+SNP 2 mM; # E6F4 DMSO+SNP 2 mM; # E6F4 817MA 0.1 μM SNP 2 mM; # E6F4 817MA 1 μM SNP 2 mM; and # E6F4 817MA 3 μM+SNP 2 mM.

FIGS. 31A and 31B show bar graphs showing data for the 817MA effect on cell viability: no SNP. FIG. 31A, shows data for 817MA 3 days. FIG. 31B, shows data for 817MA 3 weeks. Left to right, in groups of four bars, are data for non-AD, AD mixed clonal, AD single clonal and AD single clonal. From left to right each bar is for: G2 DMSO+SNP 0 mM; G2 817MA 0.1 μM SNP 0 mM; G2 817MA 1 μM SNP 0 mM; G2 817MA 3 μM+SNP 0 mM; HReN30 DMSO+SNP 0 mM; HReN30 817MA 0.1 μM SNP 0 mM; HReN30 817MA 1 μM SNP 0 mM; HReN30 817MA 3 μM+SNP 0 mM; # D4 DMSO+SNP 0 mM; # D4 817MA 0.1 μM SNP 0 mM; # D4 817MA 1 μM SNP 0 mM; # D4 817MA 3 μM+SNP 0 mM; # E6F4 DMSO+SNP 0 mM; # E6F4 817MA 0.1 μM SNP 0 mM; # E6F4 817MA 1 μM SNP 0 mM; and # E6F4 817MA 3 μM+SNP 0 mM.

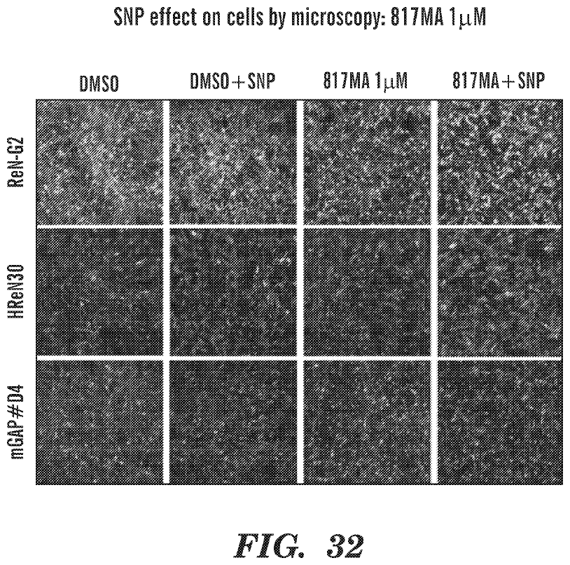

FIG. 32 shows a series of microscope images illustrating the SNP effect on cells. The images are arranged in an array. The columns from left to right are of DMSO, DMSO+SNP, 817MA 1 μM and 817MA+SNP. The rows from top to bottom are of ReN-G2, HReN30 and mGAP # D4.

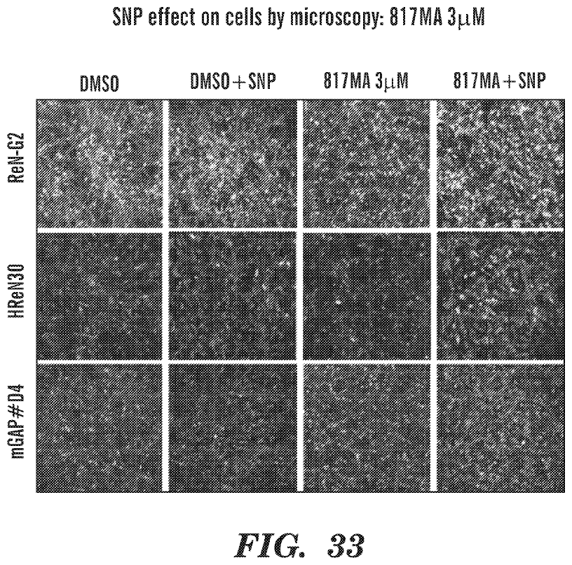

FIG. 33 shows a second series of microscope images illustrating the SNP effect on cells. The images are arranged in an array. The columns from left to right are of DMSO, DMSO+SNP, 817MA 3 μM and 817MA+SNP. The rows from top to bottom are of ReN-G2, HReN30 and mGAP # D4.

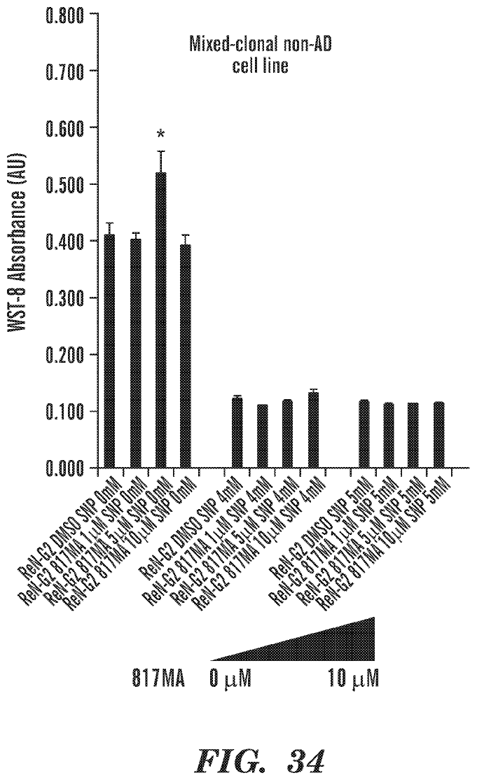

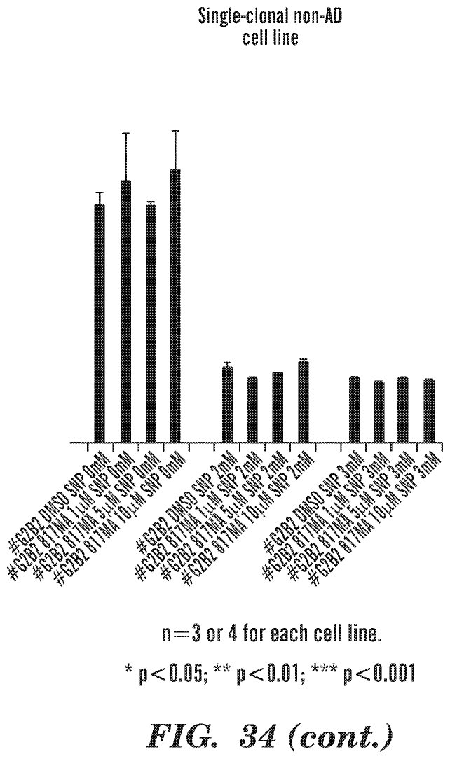

FIG. 34 shows a bar graph of data for the effect of a four-day treatment with 817MA on SNP-induced toxicity in non-AD cells. The 12 bars on the left are data of mixed-clonal non-AD cell line. The 12 bars on the right are data of single-clonal non-AD cell line. n=3 or 4 for each cell line. From left to right each bar is for: ReN-G2 DMSO SNP 0 mM; ReN-G2 817MA 1 μM SNP 0 mM; ReN-G2 817MA 5 μM SNP 0 mM; ReN-G2 817MA 10 μM SNP 0 mM; ReN-G2 DMSO SNP 4 mM; ReN-G2 817MA 1 μM SNP 4 mM; ReN-G2 817MA 5 μM SNP 4 mM; ReN-G2 817MA 10 μM SNP 4 mM; ReN-G2 DMSO SNP 5 mM; ReN-G2 817MA 1 μM SNP 5 mM; ReN-G2 817MA 5 μM SNP 5 mM; ReN-G2 817MA 10 μM SNP 5 mM; # G2B2 DMSO SNP 0 mM; # G2B2 817MA 1 μM SNP 0 mM; # G2B2 817MA 5 μM SNP 0 mM; # G2B2 817MA 10 μM SNP 0 mM; # G2B2 DMSO SNP 2 mM; # G2B2 817MA 1 μM SNP 2 mM; # G2B2 817MA 5 μM SNP 2 mM; # G2B2 817MA 10 μM SNP 2 mM; # G2B2 DMSO SNP 3 mM; # G2B2 817MA 1 μM SNP 3 mM; # G2B2 817MA 5 μM SNP 3 mM; and # G2B2 817MA 10 μM SNP 3 mM. n=3 or 4 for each cell line. *p<0.05, **p<0.01, ***p<0.001.

All patents and other publications identified in the specification and examples are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

1. A method for testing a therapeutic efficacy of a candidate substance for a prevention or treatment agent of a neurodegenerative disorder, the method comprising:
a. optionally culturing immune or immune-like cells expressing full-length human CD33;
b. treating said CD33 expressing immune or immune-like cells with said candidate substance at relevant concentration;
c. treating said CD33 expressing immune or immune-like cells with amyloid-β (Aβ) at relevant concentration;
d. measuring intracellular levels of Aβ in said CD33 expressing cells;
such that higher levels of Aβ in CD33 cells treated with the candidate substance, relative to negative control, would indicate the therapeutic efficacy.

2. The method of claim 1 wherein the immune or immune-like cells are microglial cells.

3. A method for testing the therapeutic efficacy of a candidate substance for a prevention or treatment agent of a neurodegenerative disorder, the method comprising:
a. optionally culturing immune or immune-like cells expressing full-length human CD33;
b. treating said CD33 expressing immune or immune-like cells with said candidate substance at relevant concentration;
c. treating said CD33 expressing immune or immune-like cells with lipopolysaccharide;
d. measuring levels of pro-inflammatory cytokines in culture media of said CD33 expressing cells;
such that lower levels of pro-inflammatory cytokines in the culture media of CD33 cells treated with the candidate substance, relative to negative control, would indicate the therapeutic efficacy.

4. The method of claim 3 wherein the immune or immune-like cells are microglial cells.

5. The method of claim 1, wherein the neurodegenerative disorder is selected from, but not exclusive thereof, Alzheimer's disease, mild cognitive impairment, Parkinson's disease, dementia, schizophrenia, amyotrophic lateral sclerosis, Huntington's disease and multiple sclerosis.

6. A method for testing a binding interaction of a substance with human CD33, the method comprising:
a. optionally culturing immune or immune-like cells that express full length human CD33;
b. treating said CD33 expressing immune or immune-like cells with said substance at relevant concentration;
c. measuring a read-out of the binding interaction using a standard assay;
such that a higher read-out in CD33 cells treated with the substance, relative to negative control, would indicate the binding interaction.

7. The method of claim 6 wherein the immune or immune-like cells are microglial cells.

8. A pharmaceutical composition for modulating a function of microglial cells, which comprises 1-(3-(2-(1-benzothiophen-5-yl)ethoxy)propyl)azetidin-3-ol or a salt thereof.

9. A method for modulating a function of microglial cells, comprising: administering to a patient in need thereof 1-(3-(2-(1-benzothiophen-5-yl)ethoxy)propyl)azetidin-3-ol or a salt thereof.

10. The method according to claim 9, wherein the patient has Alzheimer's disease, mild cognitive impairment, Parkinson's disease, dementia, schizophrenia, amyotrophic lateral sclerosis, Huntington's disease or multiple sclerosis.

11. The method according to claim 8, wherein the modulating a function of microglial cells is enhancing phagocytosis or inhibiting cytokine production of microglial cells.

12. A method for modulating a function of microglial cells, comprising: administering to a patient in need thereof a substance identified by the method of claim 1.

13. The method according to claim 12, wherein the substance is 1-(3-(2-(1-benzothiophen-5-yl)ethoxy)propyl)azetidin-3-ol or a salt thereof.

14. The method according to claim 12, wherein the patient has Alzheimer's disease, mild cognitive impairment, Parkinson's disease, dementia, schizophrenia, amyotrophic lateral sclerosis Huntington's disease or multiple sclerosis.

15. A method to treat or prevent a neurodegenerative disease comprising administering to a patient a therapeutic dose or dosages of compositions that are identified as a possible drug in the method of claim 1.

16. The method of claim 15 wherein the neurodegenerative disease is Alzheimer's disease.
